SEQUENCE LISTING (5 sequences)

SEQ ID NO: 1 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 2 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 3 - DNA, 1619 bp, Homo sapiens
ggggagtctaggtgcagctgccggatccttcagcgtctgcatctcggcgtcgccccgcgtaccgtcgcccggctctccgccgctctcccggggtttcggggcacttgggtcccacagtctggtcctgcttcaccttcccctgacctgagtagtcgccatggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatttctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagactgatctgtttaacatcaggaaggagtttaggaagaattttgccacctctctttattccatgattaagggagatacatctggggactataagaaagctcttctgctgctctgtggagaagatgactaacgtgtcacggggaagagctccctgctgtgtgcctgcaccaccccactgccttccttcagcacctttagctgcatttgtatgccagtgcttaacacattgccttattcatactagcatgctcatgaccaacacatacacgtcatagaagaaaatagtggtgcttctttctgatctctagtggagatctctttgactgctgtagtactaaagtgtacttaatgttactaagtttaatgcctggccattttccatttatatatattttttaagaggctagagtgcttttagccttttttaaaaactccatttatattacatttgtaaccatgatactttaatcagaagcttagccttgaaattgtgaactcttggaaatgttattagtgaagttcgcaactaaactaaacctgtaaaattatgatgattgtattcaaaagattaatgaaaaataaacatttctgtccccctgataaaaaaaaaaaaaaaaaaaaaaaaaaaa

SEQ ID NO: 4 - protein, 258 aa, Homo sapiens
MAAPAPGLISVFSSSQELGAALAQLVAQRAACCLAGARARFALGLSGGSLVSMLARELPAAVAPAGPASLARWTLGFCDERLVPFDHAESTYGLYRTHLLSRLPIPESQVITINPELPVEEAAEDYAKKLRQAFQGDSIPVFDLLILGVGPDGHTCSLFPDHPLLQEREKIVAPISDSPKPPPQRVTLTLPVLNAARTVIFVATGEGKAAVLKRILEDQEENPLPAALVQPHTGKLCWFLDEAAARLLTVPFEKHSTL

SEQ ID NO: 5 - DNA, 1034 bp, Homo sapiens
ctcctccccgccgccgccctcgccatggccgcgccggccccgggcctcatctcggtgttctcgagttcccaggagctgggtgcggcgctagcgcagctggtggcccagcgcgcagcatgctgcctggcaggggcccgcgcccgtttcgcgctcggcttgtcgggcgggagcctcgtctcgatgctagcccgcgagctacccgccgccgtcgcccctgccgggccagctagcttagcgcgctggacgctgggcttctgcgacgagcgcctcgtgcccttcgatcacgccgagagcacgtacggcctctaccggacgcatcttctctccagactgccgatcccagaaagccaggtgatcaccattaaccccgagctgcctgtggaggaggcggctgaggactacgccaagaagctgagacaggcattccaaggggactccatcccggttttcgacctgctgatcctgggggtgggccccgatggtcacacctgctcactcttcccagaccaccccctcctacaggagcgggagaagattgtggctcccatcagtgactccccgaagccaccgccacagcgtgtgaccctcacgctacctgtcctgaatgcagcacgaactgtcatctttgtggcaactggagaaggcaaggcagctgttctgaagcgcattttggaggaccaggaggaaaacccgctgcccgccgccctggtccagccccacaccgggaaactgtgctggttcttggacgaggcggccgcccgcctcctgaccgtgcccttcgagaagcattccactttgtagctggccagagggacgccgcagctgggaccaggcacgcggcccatggggctgggcccctgctggccgccactctccgggctctcctttcaaaaagccacgtcgtgctgctgctggaagccaacagcctccggccagcagccctacccggggctcaacacacaggctgtggctctggacatccggatattaaaaggagcgttgctggaaaaaaaaaaaaaaaaaaaaaaaaaaaa

(19) United States
(12) Patent Application Publication
(10) Pub. No.: US 2007/0243561 A1
(43) Pub. Date: Oct. 18, 2007

(54) PROGNOSTIC MARKERS IN CHRONIC LYMPHOCYTIC LEUKEMIA

(76) Inventors: Ben Geeraerts, Lochristi (BE); Laura Z. Rassenti, San Diego, CA (US); Fritz Offner, Melle (BE); Thomas J. Kipps, San Diego, CA (US); Dieter Deforce, Kuurne (BE)

Correspondence Address: GIFFORD, KRASS, SPRINKLE,ANDERSON & CITKOWSKI, P.C, PO BOX 7021, TROY, MI, 48007-7021, US

(21) Appl. No.: 11/675,958

(22) Filed: Feb. 16, 2007

Related U.S. Application Data
(60) Provisional application No. 60/773,926, filed on Feb. 16, 2006.

(51) Int. Cl. G01N 33/53

(52) U.S. Cl. 435/7.4

(57) ABSTRACT

Methods and compositions for detecting a form of chronic lymphocytic leukemia in a subject are described which allow for distinguishing more aggressive forms of the disease from less aggressive forms. Particularly described methods include assaying a sample obtained from the subject having or suspected of having chronic lymphocytic leukemia for a marker selected from a vimentin cleavage product, annexin 5 and 6-phosphogluconolactonase. A combination of any of these markers may be assayed in particular embodiments of a method according to the present invention. In particular embodiments, detection of an intermediate weight vimentin cleavage product is indicative of a less aggressive form of CLL associated with good prognosis.

### REFERENCE TO RELATED APPLICATION

This application claims priority of U.S. Provisional Patent Application 60/773,926, filed Feb. 16, 2006, the entire content of which is incorporated by reference herein.

### FIELD OF THE INVENTION

The present invention provides compositions and methods relating generally to detection of a form of leukemia. In particular embodiments, the present invention relates to detection of vimentin, annexin 5 and/or 6-phosphogluconolactonase in a subject having or suspected of having chronic lymphocytic leukemia.

### BACKGROUND OF THE INVENTION

B-cell neoplasms are characterized by diverse predisposing factors, symptoms and prognoses. These diseases are classified according to numerous criteria including, for example, differences in morphological characteristics of affected cells, immunophenotype, genetic features and clinical features. Accurate diagnosis and prognosis in cases of B-cell neoplasm has important implications for patient treatment.

Chronic lymphocytic leukemia (CLL) is a B-cell neoplasm which has a highly varied course in affected subjects. CLL can be categorized in at least two different subgroups, with different clinical outcome [Wiestner, A., Rosenwald, A., Barry, T. S., Wright, G., et al., Blood 2003, 101, 4944-4951; Hamblin, T., Ann Hematol 2002, 81, 99-303. Epub 2002 May 2018; Rozman, C. and Montserrat, E., N Engl J Med 1995, 333, 1052-1057; Chen, L., Widhopf, G., Huynh, L., Rassenti, L., et al., Blood 2002, 100, 4609-4614.] Some patients have an indolent disease with little need for therapeutic intervention, while other patients show a more aggressive clinical course requiring therapeutic intervention. Less aggressive forms of CLL are generally monitored but not treated since the risks associated with therapy can outweigh the benefits. However, other forms of the disease can progress rapidly resulting in uncomfortable symptoms, repeated serious infections and death, warranting aggressive therapeutic intervention such as bone marrow transplant, chemotherapy and monoclonal antibody therapy. The significant differences in clinical outcome associated with diverse forms of CLL make it especially important to be able to distinguish more aggressive forms of the disease from less aggressive forms.

Several markers have been identified which may have prognostic value in CLL including beta-2-microglobulin, immunoglobulin variable region heavy chain gene (IgV_{H}) mutational status, ZAP-70, CD-38, lipoprotein lipase and certain chromosomal abnormalities such as 11q, 13q and 17p deletions. (Damle, R. N., Wasil, T., Fais, F., Ghiotto, F., et at., Blood 1999, 94, 1840-1847; Hamblin, T. J., Davis, Z., Gardiner, A., Oscier, D. G. and Stevenson, F. K., Blood 1999, 94, 1848-1854; Rassenti, L. Z., Huynh, L., Toy, T. L., Chen, L., et al., N Eng J Med 2004, 351, 893-901; Krober, A., Seiler, T., Benner, A., Bullinger, L., et al., Blood 2002, 100, 1410-1416; Oscier, D. G., Gardiner, A. C., Mould, S. J., Glide, S., et al., Blood 2002, 100, 1177-1184; Rassenti, L. Z., Huynh, L., Toy, T. L., Chen, L., et al., N Engl J Med 2004, 351, 893-901; Orchard, J. A., Ibbotson, R. E., Davis, Z., Wiestner, A., et al., Lancet 2004, 363, 105-111; Wiestner, A., Rosenwald, A., Barry, T. S., Wright, G., et al., Blood 2003, 101, 4944-4951). However, no single marker appears to be definitive in classifying CLL patients and it is possible that clonal evolution of cells involved in CLL results in heterogeneous populations of cells which change over time.

Thus, there is a continuing need for new markers of CLL and particularly for prognostic markers of CLL.

### SUMMARY OF THE INVENTION

A method of detecting a form of chronic lymphocytic leukemia in a subject is provided which includes assaying a sample obtained from the subject having or suspected of having chronic lymphocytic leukemia for a marker selected from a vimentin cleavage product, annexin 5 and 6-phosphogluconolactonase. A combination of any of these markers may be assayed in particular embodiments of a method according to the present invention. An assay result is obtained which is indicative of a form of chronic lymphocytic leukemia in the subject.

In certain embodiments, detecting a vimentin cleavage product includes contacting the sample with a vimentin cleavage product binding agent.

Optionally, a method of detecting a form of chronic lymphocytic leukemia in a subject further includes detecting uncleaved vimentin.

Assaying the sample optionally includes detecting annexin 5 protein, annexin 5 nucleic acid, 6-phosphogluconolactonase protein, a 6-phosphogluconolactonase nucleic acid. Also provided are methods in which combinations of these markers are assayed.

Methods for detecting a form of chronic lymphocytic leukemia in a subject according to the present invention includes assay by techniques illustratively including ELISA, flow cytometry, immunoblot, immunoprecipitation, immunocytochemistry, radioimmunoassay, RT-PCR, Northern blot hybridization, dot blot hybridization, RNAse protection, mass spectrometry, enzyme assay, or combinations of any of these.

In particular embodiments, an assay result is compared with a standard.

In further particular embodiments, a second marker is assayed in methods for detecting a form of chronic lymphocytic leukemia in a subject. Illustrative second markers include beta-2-microglobulin, immunoglobulin variable region heavy chain gene, ZAP-70, CD-38, lipoprotein lipase, a chromosomal abnormality such as an 11q, 13q and/or 17p deletion associated with chronic lymphocytic leukemia; or a combination of any of these.

In one embodiment, a method of detecting a form of chronic lymphocytic leukemia in a subject includes contacting a sample obtained from the subject having or suspected of having chronic lymphocytic leukemia with a vimentin cleavage product binding agent under binding conditions. The sample is assayed to detect the presence of a complex of a vimentin cleavage product binding agent and a vimentin cleavage product to determine the presence of the vimentin cleavage product. For example, such assays include ELISA, flow cytometry, immunoblot, immunoprecipitation, immunocytochemistry, radioimmunoassay, RT-PCR, Northern blot hybridization, dot blot hybridization, RNAse protection, mass spectrometry, enzyme assay, or combinations of any of these. Detection of the presence of the vimentin cleavage product is indicative of a form of chronic lymphocytic leukemia in the subject characterized by a less aggressive clinical course and is therefore associated with a relatively positive prognosis.

In a further particular embodiment, the sample obtained from the subject having or suspected of having chronic lymphocytic leukemia is contacted with a binding agent recognizing multiple forms of vimentin, including full-length vimentin, IMW vimentin and LMW vimentin, where full-length vimentin IMW vimentin and LMW vimentin are described as “total” vimentin.

A specific embodiment includes detection of a ratio of IMW vimentin cleavage product to total vimentin wherein a ratio of about 0.33 or greater is indicative of favorable prognosis of chronic lymphocytic leukemia in the subject.

The vimentin cleavage product binding agent recognizing a vimentin cleavage product, such as IMW vimentin, and the total vimentin binding agent are identical in certain embodiments. For example, a vimentin cleavage product binding agent is optionally an antibody recognizing an epitope common to the vimentin cleavage product, and uncleaved vimentin, and/or LMW vimentin.

In further embodiments, a vimentin cleavage product binding agent used in an inventive method recognizes an epitope specific to the vimentin cleavage product and does not substantially bind uncleaved vimentin. In a particular embodiment, an IMW vimentin cleavage product binding agent used in an inventive method recognizes an epitope specific to IMW vimentin cleavage product and does not substantially bind uncleaved vimentin or LMW vimentin.

Also provided is a method of detecting a form of chronic lymphocytic leukemia in a subject including assaying a sample obtained from the subject having or suspected of having chronic lymphocytic leukemia for annexin 5 and/or 6-phosphogluconolactonase, to obtain a first assay result. The first assay result is compared with a standard in a particular embodiment. Detection of enrichment of annexin 5 and/or 6-phosphogluconolactonase in the sample is indicative of a form of chronic lymphocytic leukemia having a poor prognosis, thereby detecting a form of chronic lymphocytic leukemia in the subject.

In further embodiments, a kit for detection of a form of chronic lymphocytic leukemia in a subject is provided according to the present invention which includes a binding agent for substantially specific binding to a substance selected from the group consisting of: a vimentin cleavage product, uncleaved vimentin, annexin 5,6-phosphogluconolactonase, an annexin 5 nucleic acid, a 6-phosphogluconolactonase nucleic acid, and a combination of any of these. Instructions for use in detection of a form of chronic lymphocytic leukemia in a subject are also included.

Optionally, a kit according to a further embodiment of the present invention includes a binding agent for specifically detecting beta-2-microglobulin, immunoglobulin variable region heavy chain gene, ZAP-70, CD-38, lipoprotein lipase, a chromosomal abnormality associated with chronic lymphocytic leukemia, or a combination of any of these.

### BRIEF DESCRIPTION OF THE DRAWINGS

FIG. 1 is a xerographic reproduction of a representative digitized 2-DE gel image from a processed sample obtained from a CLL patient;

FIG. 2 is a graphic illustration of a dendrogram showing patterns obtained from different patients which are marked designating patient number, mutational status, and ZAP-70 status;

FIG. 3 is a graphic representation showing the relationship between the relative normalized expression levels of a particular protein in each CLL sample and the CLL subgroup these samples are assigned to for several spots resulting from the statistical analysis;

FIG. 4A is a xerographic reproduction of an image showing vimentin immunodetection by Western blot analysis of 2-DE separated soluble proteins obtained from a CLL sample from a UM-CLL subject;

FIG. 4B is a xerographic reproduction of an image showing vimentin immunodetection by Western blot analysis of 2-DE separated soluble proteins obtained from a CLL sample from an M-CLL subject;

FIG. 4C is a graph illustrating quantitation of spots corresponding to HMW, IMW and LMW vimentin in samples obtained from an M-CLL subject and in a UM-CLL subject;

FIG. 5A is a xerographic reproduction of an image of a Western blot analysis of vimentin and annexin expression in patients with different mutational and ZAP-70 status;

FIG. 5B is a graphic illustration of an expression ratio of IMW vimentin/total vimentin in all samples according to their mutational status;

FIG. 5C is a graphic illustration of the normalized expression ratio of Annexin A5 in all samples according to their mutational status;

FIG. 5D is a xerographic reproduction of an image of the gel shown in FIG. 5A with a superimposed graphic indicating molecular weights of vimentin and vimentin cleavage products.

### DETAILED DESCRIPTION OF THE PREFERRED EMBODIMENTS

Methods and compositions for detecting a form of chronic lymphocytic leukemia in a subject are provided according to the present invention. In particular embodiments, methods and compositions according to the present invention are described which allow a user to distinguish between forms of chronic lymphocytic leukemia having good and poor prognosis. Provided methods and compositions are useful in contributing to data available to clinicians for use in considering therapeutic options, for example. Methods and compositions according to the present invention also have utility in research directed toward molecular mechanisms underlying chronic lymphocytic leukemia and the clinical course of the disease.

Inventive methods are described herein which include detection of particular markers in order to distinguish between forms of chronic lymphocytic leukemia having different prognoses. Particular markers include a vimentin cleavage product, annexin 5 and/or 6-phosphogluconolactonase. Specific embodiments of methods according to the present invention include detection of enrichment of annexin 5 and/or 6-phosphogluconolactonase protein and/or RNA.

A method of detecting a form of chronic lymphocytic leukemia in a subject is provided which includes assaying a sample obtained from a subject having or suspected of having chronic lymphocytic leukemia for a vimentin cleavage product, annexin 5 and/or 6-phosphogluconolactonase. Assaying a sample produces an assay result which is qualitative or quantitative depending on the desired result and/or assay method employed. Combinations of a vimentin cleavage product, annexin 5 and/or 6-phosphogluconolactonase are optionally detected in particular embodiments. Detection of one or more of these markers is found to be indicative of chronic lymphocytic leukemia having a particular prognosis.

A sample obtained from a subject for use in a method according to embodiments of the present invention includes a B-cell, such as a normal and/or neoplastic B-cell. Suitable samples illustratively include, but are not limited to, blood, a blood fraction such as plasma; lymph fluid; and biopsy, necropsy and/or transplant tissues illustratively including lymphatic tissue such as a lymph node, spleen, bone marrow, tonsils, diffuse lymphoid tissue and Peyer's patches.

Detection of a marker for a form of CLL according to embodiments of inventive methods includes detection of presence of the marker and/or quantity of the marker. An assay to detect a marker for a form of CLL includes assay for characteristics specific to the marker such as physical, chemical and/or functional properties of the marker. Assays illustratively include marker binding assays, assays for marker protein function and mass spectrometry, for example.

In particular embodiments, detection of a vimentin cleavage product, annexin 5 and/or 6-phosphogluconolactonase includes contacting a sample with a binding agent characterized by substantially specific binding to a vimentin cleavage product, annexin 5 and/or 6-phosphogluconolactonase target.

The term “binding agent” refers to an agent characterized by substantially specific binding to a specified substance. In particular embodiments of the present invention, a binding agent binds substantially specifically to a marker for a form of CLL, such as a vimentin cleavage fragment, an IMW vimentin cleavage fragment, annexin 5 and/or 6-phosphogluconolactonase. A suitable binding agent illustratively includes, but is not limited to, an antibody, a nucleic acid probe and/or primer, a nucleic acid and/or peptide aptamer and a synthetic small molecule chemical compound.

The phrase “substantially specific” as used herein in reference to binding of a binding agent to a specified substance, such as a specified marker, refers to binding of the binding agent to the specified substance without substantial binding to other substances present in a sample to be assayed for presence of the specified substance. In particular embodiments, a binding agent binds to a vimentin cleavage fragment, an IMW vimentin cleavage fragment, uncleaved vimentin, annexin 5 and/or 6-phosphogluconolactonase without substantially binding to another substance present in a sample obtained from a subject.

Substantially specific binding of a binding agent can be demonstrated by various techniques including, for example, ELISA, and other immunoassays for antibodies, and hybridization under high stringency hybridization and/or washing conditions for nucleic acids.

The term “binding” refers to a physical or chemical interaction between the binding agent and the target. Binding includes, but is not limited to, ionic bonding, non-ionic bonding, covalent bonding, hydrogen bonding, hydrophobic interaction, hydrophilic interaction, and Van der Waals interaction.

Compositions and methods are provided according to the present invention wherein a binding agent is an antibody in particular embodiments. The term “antibody” is used herein in its broadest sense and includes single antibodies and mixtures of antibodies characterized by substantially specific binding to an antigen. An antibody provided according to compositions and methods is illustratively a polyclonal antibody, a monoclonal antibody, a chimeric antibody, a humanized antibody, and/or an antigen binding antibody fragment, for example. The term antibody refers to a standard intact immunoglobulin having four polypeptide chains including two heavy chains (H) and two light chains (L) linked by disulfide bonds in particular embodiments. Antigen binding antibody fragments illustratively include an Fab fragment, an Fab′ fragment, an F(ab′)2 fragment, an Fd fragment, an Fv fragment, an scFv fragment and a domain antibody (dAb), for example. In addition, the term antibody refers to antibodies of various classes including IgG, IGM, IgA, IgD) and IgE, as well as subclasses, illustratively including for example human subclasses IgG1, IgG2, IgG3 and IgG4 and murine subclasses IgG1, IgG2, IgG2a. IgG2b, IgG3 and IgGM, for example.

In particular embodiments, an antibody which is characterized by substantially specific binding has a dissociation constant, K_{d}, less than about 10⁻⁷M, such as less than about 10⁻⁸M, less than about 10⁻⁹M or less than about 10⁻¹⁰M, or less depending on the specific composition. Binding affinity of an antibody can be determined by Scatchard analysis such as described in P. J. Munson and D. Rodbard, Anal. Biochem., 107:220-239, 1980.

A binding agent is a nucleic acid in particular embodiments.

A nucleic acid binding agent is used in any of various techniques to detect annexin 5 and/or 6-phosphogluconolactonase nucleic acids illustratively including RT-PCR, Northern blot hybridization, dot blot hybridization, RNAse protection, and a combination of any of these.

For example, a binding agent is optionally a primer and/or probe which hybridizes substantially specifically to an annexin 5 or 6-phosphogluconolactonase nucleic acid such as a cDNA or mRNA.

The term “primer” refers to a single stranded DNA oligonucleotide complementary to a target annexin 5 or 6-phosphogluconolactonase nucleic acid and capable of acting as a point of initiation of a primer extension product when placed under conditions in which such a primer extension product complementary to the target nucleic acid is produced. Such conditions are known in the art and include, for example, presence of a DNA polymerase, suitable pH and temperature. A primer may be double stranded where the two strands are separated prior to use. A primer generally has a length of about 10-50 nucleotides depending on the conditions under which the reaction will occur, the composition of the target and the like. Primers are useful as binding agents in assays illustratively including PCR amplification and RNA extension, for example. Methods of design and synthesis of primers and their use in nucleic acid detection are known in the art as detailed in references cited herein such as J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002.

The term “probe” refers to a single stranded sequence specific oligonucleotide or polynucleotide which is complementary to a target sequence of annexin 5 or 6-phosphogluconolactonase. A probe ranges in size depending on the particular application and target and is generally in the range of, but not limited to, about 10 nucleotides to one or more kilobases. A probe is used in any of various assay techniques to detect an annexin 5 or 6-phosphogluconolactonase nucleic acid illustratively including Northern blot hybridization, dot blot hybridization, RNAse protection and other hybridization assays. Details of such assays are described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002, for example.

The term “nucleic acid” as used herein refers to RNA or DNA molecules having more than one nucleotide in any form including single-stranded, double-stranded, oligonucleotide or polynucleotide. The term “nucleotide sequence” is used to refer to the ordering of nucleotides in an oligonucleotide or polynucleotide in a single-stranded form of nucleic acid.

The terms “duplex” and “double-stranded” are used to refer to nucleic acids characterized by binding interaction of complementary nucleotide sequences. A duplex includes a “sense” strand and an “antisense” strand. Such duplexes include RNA/RNA, DNA/DNA or RNA/DNA types of duplexes. A duplex may be formed from two nucleotide sequences which are otherwise unconnected. Alternatively, a duplex may be formed by a single-stranded nucleic acid where the single-stranded nucleic acid has substantially complementary sense and antisense regions. Such a nucleic acid forms a “hairpin” conformation when the substantially complementary sense and antisense regions are hybridized to form a duplex.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′. It will be recognized by one of skill in the art that two complementary nucleotide sequences include a sense strand and an antisense strand.

The degree of complementarity, also called homology, between nucleic acid strands significantly affects binding of the strands to each other. An antisense strand which is substantially complementary to a sense strand hybridizes to the sense strand under high stringency hybridization conditions.

The term “hybridization” refers to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “hybridizes substantially specifically” refer to hybridization of a particular nucleic acid to a target nucleic acid without substantial hybridization to nucleic acids other than the target nucleic acid.

The term “oligonucleotide” is used herein to describe a nucleotide sequence having from 2-100 linked nucleotides, while the term “polynucleotide” is used to describe a nucleotide sequence having more than 100 nucleotides.

A binding agent binds substantially specifically to a vimentin cleavage fragment, an IMW vimentin cleavage fragment, uncleaved vimentin, annexin 5 and/or 6-phosphogluconolactonase under suitable binding conditions. Suitable binding conditions depend on the identity and type of the binding agent and determination of suitable binding conditions is performed without undue experimentation by one of ordinary skill in the art. For example, binding conditions for antibodies are established by routine ELISA, immunoblot, immunocytochemistry and the like varying concentration of antibody, buffer composition, blockers of non-specific binding, temperature, and cell permeabilization methods, for example. Binding conditions for nucleic acid binding agents are similarly established by routine calculation of hybrid melting temperature, Tm, and varying concentration of probe, buffer composition, blockers of non-specific binding, temperature, and template nucleic acid concentration, for example. Factors affecting binding of binding agents are well-known in the art and are described in detail in E. Harlow and D. Lane, Antibodies: A Laboratory Manual, Cold Spring Harbor Laboratory Press, 1988; F. Breitling and S. Düibel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; Ormerod, M. G., and J. Sambrook and D. W. Russell, Molecular Cloning: A Laboratory Manual, Cold Spring Harbor Laboratory Press, 3^{rd} Ed., 2001. In particular embodiments, suitable binding conditions include, but are not limited to, conditions which approximate physiological conditions with respect to one or more of: pH and ionic strength, for example. Binding conditions for antibody binding agents are optionally in the range of about 4° C.-40° C., but may be outside this range depending on the identity of the antibody and specific reaction conditions.

Detailed description of suitable binding conditions for a binding agent such as an antibody, aptamer or nucleic acid are described, for example, in F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3^{rd} Ed., 2001.

Particular methods of assay using a binding agent are known in the art and illustratively include enzyme-linked immunosorbent assay (ELISA), flow cytometry, immunoblot, immunoprecipitation, immunocytochemistry, radioimmunoassay, RT-PCR, Northern blot hybridization, dot blot hybridization, RNAse protection, and a combination of any of these. Assay methods may be used to obtain qualitative and/or quantitative results. Specific details of suitable assay methods for both qualitative and quantitative assay of a sample are described in standard references, illustratively including E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering Methods and Protocols, Methods in Molecular Biology, Humana Press, 2003; F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; Ormerod, M. G., Flow Cytometry: a practical approach, Oxford University Press, 2000; Givan, A. L., Flow Cytometry: first principles, Wiley, New York, 2001; Gorczyca, W., Flow Cytometry in Neoplastic Hematology: morphologic-immunophenotypic correlation, Taylor & Francis, 2006; and J. Sambrook and D. W. Russell, Molecular Cloning: A Laboratory Manual, Cold Spring Harbor Laboratory Press, 3^{rd} Ed., 2001.

In particular embodiments, a sample obtained from a subject an assay for a vimentin cleavage product, uncleaved vimentin, annexin 5 and/or 6-phosphogluconolactonase includes use of a mass spectrometry technique. For example, a vimentin cleavage product, uncleaved vimentin, annexin 5 and/or 6-phosphogluconolactonase can be ionized using an ionization method such as electrospray ionization (ESI), matrix-assisted laser desorption/ionization (MALDI) or surface enhanced laser desorption/ionization (SELDI). Mass analysis is conducted using, for example, time-of-flight (TOF) mass spectrometry or Fourier transform ion cyclotron resonance mass spectrometry. Mass spectrometry techniques are known in the art and exemplary detailed descriptions of methods for protein and/or peptide assay are found in Li J., et al., Clin Chem., 48(8):1296-304, 2002; Hortin, G. L., Clinical Chemistry 52: 1223-1237, 2006; Hortin, G. L., Clinical Chemistry 52: 1223-1237, 2006; A. L. Burlingame, et al. (Eds.), Mass Spectrometry in Biology and Medicine, Humana Press, 2000; and D. M. Desiderio, Mass Spectrometry of Peptides, CRC Press, 1990.

In further embodiments, an assay according to the present invention includes use of an assay technique, such as a mass spectrometry technique, in conjunction with one or more methods for generation of peptides from a sample. Peptides are illustratively generated by enzyme digestion. In a particular embodiment, particular peptides are generated including N-terminal peptides, cysteinyl peptides, methionyl peptides, phosphorylated peptides, purified by COmbined FRActional DIagonal Chromatography (COFRADIC) and analysed by a technique such as mass spectrometry. Examples of such assays are described in detail in Gevaert, K., et al., Mol Cell Proteomics, 1(11):896-903, 2002; Gevaert, K., et al., Nat. Biotechnol., 21(5):566-9, 2003; and Martens L, et al., Proteomics, 5(12):3193-204, 2005.

A vimentin cleavage product, uncleaved vimentin, annexin 5 protein and/or annexin 5 nucleic acid and/or 6-phosphogluconolactonase protein and/or 6-phosphogluconolactonase nucleic acid contained in a sample from a patient is optionally purified for assay according to a method of the present invention.

The term “purified” in the context of a sample refers to separation of a vimentin cleavage product, uncleaved vimentin, annexin 5 protein and/or annexin 5 nucleic acid and/or 6-phosphogluconolactonase protein and/or 6-phosphogluconolactonase nucleic acid in the sample from at least one other component present in the sample.

In particular embodiments, a vimentin cleavage product, uncleaved vimentin, annexin 5 protein and/or annexin 5 nucleic acid and/or 6-phosphogluconolactonase protein and/or 6-phosphogluconolactonase nucleic acid is optionally substantially purified from the sample to produce a substantially purified sample for use in an inventive assay. The term “substantially purified” refers to a vimentin cleavage product, uncleaved vimentin, annexin 5 protein and/or annexin 5 nucleic acid and/or 6-phosphogluconolactonase protein and/or 6-phosphogluconolactonase nucleic acid separated from other substances naturally present in a sample obtained from the subject so that a vimentin cleavage product, uncleaved vimentin, annexin 5 protein and/or annexin 5 nucleic acid and/or 6-phosphogluconolactonase protein and/or 6-phosphogluconolactonase nucleic acid makes up at least about 1-100% of the mass, by weight, such as about 1%, 5%, 10%, 25%, 50% 75% or greater than about 75% of the mass, by weight, of a substantially purified sample.

Sample purification is achieved by techniques illustratively including electrophoretic methods such as gel electrophoresis and 2-D gel electrophoresis; chromatography methods such as HPLC, ion exchange chromatography, affinity chromatography, size exclusion chromatography, thin layer and paper chromatography. It is appreciated that electrophoresis and chromatographic methods can also be used to separate a vimentin cleavage product, uncleaved vimentin, annexin 5 and/or 6-phosphogluconolactonase from other components in a sample in the course of performing an assay, as in, for example separation of proteins or nucleic acids in immunoblot or Northern blot assays.

In certain embodiments, an assay for a vimentin cleavage product, uncleaved vimentin, annexin 5 and/or 6-phosphogluconolactonase includes assay of function of a vimentin cleavage product, uncleaved vimentin, annexin 5 and/or 6-phosphogluconolactonase. For example, enzyme activity of 6-phosphogluconolactonase is optionally detected and/or measured. An assay for 6-phosphogluconolactonase is described in detail in Moir, R. D. and Stokes, G. B., Biochem. J. 256:69-73, 1988.

A particular finding of the present invention is that a vimentin cleavage product is enriched in a sample obtained from a subject having chronic lymphocytic leukemia characterized by a less aggressive clinical course and is therefore associated with a relatively positive prognosis.

A sample characterized by a less aggressive clinical course is also characterized by highly mutated IgVH genes. In certain embodiments, IgVH genes are characterized as highly mutated where the percent sequence homology of the IgVH genes present in a sample obtained from a subject is less than about 96-98% compared to the corresponding germ-line IgVH gene. In contrast, in such embodiments, IgVH genes are characterized as less mutated where the percent sequence homology of the IgVH genes present in a sample obtained from a subject is about 96-98% or greater compared to the corresponding germ-line IgVH gene.

In particular embodiments, IgVH genes are characterized as highly mutated where the percent sequence homology of the IgVH genes present in a sample obtained from a subject is less than about 98% compared to the corresponding germ-line IgVH gene. In contrast, IgVH genes are characterized as less mutated where the percent sequence homology of the IgVH genes present in a sample obtained from a subject is about 98% or greater compared to the corresponding germ-line IgVH gene.

Determination of the % homology between IgVH genes present in a sample obtained from a subject compared to the corresponding germ-line IgVH gene is determined by methods illustratively including PCR and nucleic acid sequencing. Particular methods for determination of the % homology between IgVH genes present in a sample obtained from a subject compared to the corresponding germ-line IgVH gene are detailed in Wiestner, A., et al., Blood, 2003, 101, 4944-4951; Davis, Z. A., et al., Blood, 2003, 102, 3075; Damle, R. N., et al., Blood, 1999, 94, 1840-1847; Hamblin, T. J., et al., Blood 1999, 94, 1848-1854; Oscier, D. G., et al., Blood 1997, 89, 4153-4160; Krober, A., et al., Blood, 2002, 100, 1410-1416; Rassenti et al., N. Engl. J. Med., 351:893-901, 2004; and U.S. Patent Application Publication 2003/0203416.

ZAP-70 is a further prognostic indicator in CLL and samples from CLL patients which contain ZAP-70 are associated with more aggressive clinical course. Assay for ZAP-70 in a sample is performed by methods illustratively including immunocytochemistry, immunoblot and PCR. Particular examples of methods of ZAP-70 assay include those described in detail in Rassenti et al., N. Eng. J. Med., 351:893-901, 2004; and U.S. Patent Application Publication 2003/0203416.

Vimentin cleavage products indicative of a less aggressive clinical course of chronic lymphocytic leukemia according to embodiments of the present inventive methods are characterized by an apparent molecular weight in the range of about 52-54 kDa, typically about 53 kDa, described herein as intermediate weight IMW) vimentin cleavage products. Uncleaved vimentin, also termed HMW vimentin herein has an apparent molecular weight of about 58 kDa. Cleavage products of vimentin designated LMW cleavage products herein have apparent molecular weights in the range of about 23-50 kDa, including specific LMW cleavage products having apparent molecular weights of about 24 kDa, 28 kDa, 45 kDa and 49 kDa.

In particular embodiments, a subject is a human subject and therefore a human vimentin cleavage product is detected in an assay method according to the present invention. The full-length amino acid and nucleic acid sequences of human vimentin have been characterized as described in detail in several references including Ferrari, S. et al., Mol. Cell Biol. 6:3614-3620, 1986; Deloukas, P. et al., Nature, 429:375-381, 2004; Gerhard et al., Genome Res., 14:2121-2127, 2004; and Sommers, C. L. et al., Cancer Res. 49:4258-4263, 1989; and as set forth as accession number P08670 in the SwissProt database version updated Jan. 23, 2007. Further, the full-length amino acid sequence of human vimentin is set forth herein as SEQ ID No. 1

In particular embodiments, cleavage products of vimentin are cleaved relative to full-length vimentin of SEQ ID No. 1 at major cleavage sites at Asp85, Asp 259 and/or Asp 429. Specific embodiments of methods according to the present invention include detection of HMW, IMW and/or LMW vimentin cleavage products, wherein HMW is full-length human vimentin, that is, amino acids 2-466 of SEQ 1) No. 1, IMW vimentin cleavage product is a vimentin cleavage product consisting of amino acids 2-429 of human vimentin of SEQ ID No. 1, and LMW vimentin cleavage products include polypeptides consisting of amino acids 86-466 or 86-429 of human vimentin of SEQ ID No. 1.

In further embodiments, methods according to the present invention include detection of HMW, IMW and/or LMW vimentin cleavage products, wherein HMW is full-length human vimentin, that is, a protein including amino acids 2-466 or a protein having high homology to SEQ ID No. 1. In such further embodiments, IMW vimentin cleavage product is a vimentin cleavage product of about 405-440 amino acids having an amino acid sequence identical to, or having high homology to, the region of human vimentin extending between about amino acids 2-450 of SEQ ID No. 1. Further, in such embodiments, LMW vimentin cleavage products include vimentin cleavage products of about 360-400 amino acids having an amino acid sequence identical to, or having high homology to, the region of human vimentin extending between about amino acids 66-466 of human vimentin of SEQ ID No. 1 and/or vimentin cleavage products of about 320-360 amino acids having an amino acid sequence identical to, or having high homology to, the region of human vimentin extending between about amino acids 66-449 of human vimentin of SEQ ID No. 1.

The term “high homology” in this context refers to a protein having at least about 95% identity to the referenced protein or protein region.

It is appreciated that mutations within the vimentin, annexin 5 and/or 6-phosphogluconolactonase genes of an individual may result in a functional vimentin, annexin 5 and/or a 6-phosphogluconolactonase protein having an amino acid sequence which differs from the sequences described herein. Thus, in particular embodiments, detection of vimentin, annexin 5 and/or a 6-phosphogluconolactonase according to methods of the present invention includes detection of variants of these proteins. Variants include polypeptides having mutations including one or more amino acid substitutions, deletions and/or additions compared to sequences described herein. In particular embodiments, a variant has at least about 95% identity to the unmutated protein.

For example, a variant may contain one or more “conservative” amino acid substitutions in a vimentin, annexin 5 and/or a 6-phosphogluconolactonase protein of an individual subject which have little or no detectable effect on vimentin, annexin 5 and/or 6-phosphogluconolactonase function.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan. Amino acids may also be described in terms of relative size, alanine, cysteine, aspartate, glycine, asparagine, proline, threonine, serine, valine, all typically considered to be small.

In particular embodiments, detection of the IMW vimentin cleavage products includes contacting a sample with an IMW vimentin cleavage product binding agent. An IMW vimentin cleavage product binding agent is optionally an agent which also binds to uncleaved vimentin and/or other vimentin cleavage products. In such a case, assay for detection of IMW vimentin cleavage products optionally further includes separation of vimentin and vimentin cleavage products in order to identify and/or quantify IMW vimentin cleavage products. In further particular embodiments, an IMW vimentin cleavage product binding agent is substantially specific for IMW vimentin cleavage products and does not substantially bind uncleaved vimentin and/or other vimentin cleavage products.

In addition to differences in molecular weight due to differences in the number of amino acids, full length vimentin and vimentin cleavage fragments show further differences in isoelectric point. Such differences are believed to be due to phosphorylation at various phosphorylation sites in these proteins. Phosphorylation sites in human vimentin are believed to be present at amino acids 5, 7, 8, 9, 10, 25, 26, 34, 39, 42, 47, 51, 56, 66, 72, 73, 83, 117, 420, 430, 458 and 459. In particular embodiments, an assay according to the present invention includes detection of phosphorylated vimentin and/or a phosphorylated cleavage fragment of vimentin.

In further embodiments, an inventive method further includes detecting uncleaved vimentin and/or vimentin cleavage products having higher or lower apparent molecular weights than IMW vimentin cleavage products. A ratio of detected vimentin cleavage product to uncleaved and/or total detectable vimentin is optionally determined as an indicator of a form of chronic lymphocytic leukemia.

In a particular embodiment, a method of detecting a form of chronic lymphocytic leukemia in a subject is provided which includes contacting a sample obtained from the subject having or suspected of having chronic lymphocytic leukemia with a vimentin cleavage product binding agent under suitable binding conditions. Under binding conditions a complex is formed between the binding agent and a vimentin cleavage product, if present. The presence of a complex of the vimentin cleavage product binding agent and the vimentin cleavage product is detected in order to determine the presence of the vimentin cleavage product. Optionally, the amount of the vimentin cleavage product is determined using quantitative methods.

In a further option, detection of the vimentin cleavage product in the sample is compared with a standard. For example, the amount or presence of the vimentin cleavage product is compared to the amount or presence of the cleavage product detected in a sample obtained from a healthy control subject or from the subject at an earlier time. Alternatively, the standard is developed using a population of individuals having either a form of chronic lymphocytic leukemia associated with good or poor prognosis. Thus, the assay result is indicative of a particular form of chronic lymphocytic leukemia in the subject.

In a further embodiment, an inventive assay includes contacting the sample obtained from the subject having or suspected of having chronic lymphocytic leukemia with a binding agent characterized by substantially specific binding to uncleaved vimentin and/or a non-IMW vimentin cleavage product. For example, both uncleaved and IMW vimentin are detected by a binding agent in one embodiment of such an assay. In further embodiments, both uncleaved and LMW vimentin are detected by a binding agent.

In a particular embodiment, detection of a ratio of IMW vimentin cleavage product to total vimentin of about 0.33 or greater is indicative of favorable prognosis of chronic lymphocytic leukemia in the subject.

In an embodiment in which more than one of: HMW vimentin, IMW vimentin and LMW vimentin are desired to be detected, more than one binding agent is optionally used. In a further embodiment, a single binding agent which is a binding agent for more than one type of vimentin is optionally used. For example, in particular embodiments the vimentin cleavage product binding agent is an antibody recognizing an epitope common to the vimentin cleavage product and uncleaved vimentin. Thus, illustratively in one embodiment, the IMW vimentin cleavage product binding agent and the total vimentin binding agent are identical. Alternatively, a vimentin cleavage product antibody recognizes an epitope specific to the vimentin cleavage product and does not substantially bind uncleaved vimentin.

An exemplary binding agent for use in an inventive assay to detect vimentin and/or a vimentin cleavage product in a commercially available monoclonal antibody produced in mouse known as clone V9, available from Sigma-Aldrich, St. Louis, Mo. This antibody is described in published references including Osborn, M., et al., Monoclonal antibodies specific for vimentin. J. Cell Biol. 34, 137, (1984) and Bohn, W., et al., Species-specific recognition patterns of monoclonal antibodies directed against vimentin. Exp. Cell Res. 201, 1, (1992), for example. Monoclonal V9 is believed to recognize an epitope in the “tail” region of human vimentin at or near amino acid 417, Fujita J, et al., Microbiol Immunol 2003; 47(6):447-51. Additional antibodies for detection of vimentin cleavage products include a goat polyclonal antibody S-20, available commercially from Santa Cruz Biotechnology, Inc., Santa Cruz, Calif., which recognizes an epitope near the C-terminus of human vimentin. The S-20 antibody also reacts with vimentin of non-human species including mouse, rat, cow and pig.

Annexin 5, also known as annexin 5a, annexin A5 and annexin V, is found to be enriched in a sample obtained from a subject having chronic lymphocytic leukemia characterized by a more aggressive clinical course. Such a sample is also characterized by less mutated IgVH genes, an observation associated with chronic lymphocytic leukemia characterized by a more aggressive clinical course.

In a particular embodiment, a method of detecting a form of chronic lymphocytic leukemia in a subject is provided which includes contacting a sample obtained from the subject having or suspected of having chronic lymphocytic leukemia with an annexin 5 binding agent under suitable binding conditions. A complex of the annexin 5 binding agent and the annexin 5 is detected in order to determine the presence of the annexin 5. Optionally, the amount of annexin 5 is determined using quantitative methods.

In a further option, detection of annexin 5 in the sample is compared with a standard to detect enrichment in the sample. For example, the amount or presence of annexin 5 is compared to the amount of annexin 5 detected in a sample obtained from a healthy control subject or in a sample obtained from the subject at an earlier time. In a further example, the amount or presence of annexin 5 is compared to the amount of annexin 5 detected in a sample obtained from a subject known to have a form of CLL having either a good or poor prognosis. Alternatively, the standard is developed using a population of individuals having either a form of chronic lymphocytic leukemia associated with good or poor prognosis. In yet a further embodiment, the sample obtained from the subject is assayed for a second marker indicative of a form of CLL, illustratively including ZAP-70 and/or the extent of IgVH mutation. Thus, the result of an assay for annexin 5 is indicative of a particular form of chronic lymphocytic leukemia in the subject.

Assaying a sample to detect annexin 5 enrichment includes assays to detect annexin 5 protein and/or a nucleic acid encoding annexin 5. In particular embodiments, an inventive assay includes detection of full-length annexin 5 protein or a fragment thereof. Full-length human annexin 5 is the 320 amino acid protein included herein as SEQ ID No. 2. SEQ ID No. 3 illustrates a nucleic acid sequence encoding human annexin 5.

The enzyme 6-phosphogluconolactonase is found to be enriched in a sample obtained from a subject having chronic lymphocytic leukemia characterized by a more aggressive clinical course. Such a sample is also characterized by less mutated IgVH genes, an observation associated with chronic lymphocytic leukemia characterized by a more aggressive clinical course.

In a particular embodiment, a method of detecting a form of chronic lymphocytic leukemia in a subject is provided which includes contacting a sample obtained from the subject having or suspected of having chronic lymphocytic leukemia with a 6-phosphogluconolactonase binding agent under suitable binding conditions. A complex of the 6-phosphogluconolactonase binding agent and the 6-phosphogluconolactonase is detected in order to determine the presence of the 6-phosphogluconolactonase. Optionally, the amount of 6-phosphogluconolactonase is determined using quantitative methods.

In a further option, detection of 6-phosphogluconolactonase in the sample is compared with a standard to detect enrichment in the sample. For example, the amount or presence of 6-phosphogluconolactonase is compared to the amount of 6-phosphogluconolactonase detected in a sample obtained from a healthy control subject or in a sample obtained from the subject at an earlier time. In a further example, the amount or presence of 6-phosphogluconolactonase is compared to the amount of 6-phosphogluconolactonase detected in a sample obtained from a subject known to have a form of CLL having either a good or poor prognosis. Alternatively, the standard is developed using a population of individuals having either a form of chronic lymphocytic leukemia associated with good or poor prognosis. In yet a further embodiment, the sample obtained from the subject is assayed for a second marker indicative of a form of CLL, illustratively including ZAP-70 and/or the extent of IgVH mutation. Thus, the result of an assay for 6-phosphogluconolactonase is indicative of a particular form of chronic lymphocytic leukemia in the subject.

Assaying a sample to detect 6-phosphogluconolactonase enrichment includes assays to detect 6-phosphogluconolactonase protein and/or a nucleic acid encoding 6-phosphogluconolactonase. In particular embodiments, an inventive assay includes detection of full-length 6-phosphogluconolactonase protein or a fragment thereof. Full-length human 6-phosphogluconolactonase is the 258 amino acid protein included herein as SEQ ID No. 4. SEQ ID No. 5 illustrates a nucleic acid sequence encoding human annexin 5.

Detection of annexin 5 and/or 6-phosphogluconolactonase in a method according to the present invention is optionally detection of annexin 5 and/or 6-phosphogluconolactonase RNA. In specific embodiments, an assay for detection of annexin 5 in a sample obtained from a subject includes detection of RNA encoding the annexin 5 protein of SEQ ID No. 2, a variant thereof and/or a fragment thereof. In other embodiments, an assay for detection of 6-phosphogluconolactonase in a sample obtained from a subject includes detection of RNA encoding the 6-phosphogluconolactonase protein of SEQ ID No. 4, a variant thereof and/or a fragment thereof.

In further embodiments, an assay for detection of annexin 5 in a sample obtained from a subject includes detection of the nucleic acid SEQ ID No.3 and/or a fragment thereof.

In still further embodiments, an assay for detection of 6-phosphogluconolactonase in a sample obtained from a subject includes detection of the nucleic acid of SEQ ID No.5 and/or a fragment thereof.

It is appreciated that due to the degeneracy of the genetic code, one or more nucleotides in a nucleotide sequence may differ from SEQ ID Nos. 3 and 5 and still encode the proteins of SEQ ID Nos. 2 and 4, respectively. Thus, assays for annexin 5 or 6-phosphogluconolactonase which encode the proteins of SEQ ID Nos. 2 and 4 but which are not identical to SEQ ID Nos. 3 and 5 are considered to be within the scope of the present invention. Further, as noted above, one or more conservative amino acid substitutions may occur in a protein without substantial effect on the function of the protein. Thus, one of ordinary skill in the art will appreciate that methods including detection of a nucleic acid encoding annexin 5,6-phosphogluconolactonase and variants thereof are within the scope of the present invention. Nucleic acids encoding a variant of annexin 5 or 6-phosphogluconolactonase have one or more nucleotide substitutions, deletions and/or additions compared to SEQ ID Nos. 3 and 5.

An assay result is optionally compared with a standard indicative of enrichment of annexin 5 and/or 6-phosphogluconolactonase in the sample. For example, a standard is illustratively an assay result of an assay for annexin 5 and/or 6-phosphogluconolactonase performed on one or more subjects having an aggressive or non-aggressive form of CLL. A standard is optionally an assay result of an assay annexin 5 and/or 6-phosphogluconolactonase performed on one or more healthy subjects. Thus, detection of enrichment of annexin 5 and/or 6-phosphogluconolactonase is indicative of an aggressive form of chronic lymphocytic leukemia in the subject.

In further embodiments, assaying a sample obtained from a subject having or suspected of having chronic lymphocytic leukemia includes assay for a second marker associated with a form of chronic lymphocytic leukemia. For example, beta-2-microglobulin, immunoglobulin variable region heavy chain gene mutation, ZAP-70, CD-38, lipoprotein lipase are each found to have some association with a form of chronic lymphocytic leukemia. Assays and specific reagents for detection of these markers of CLL are described herein and in Wiestner, A., et al., Blood, 2003, 101, 4944-4951; Davis, Z. A., et al., Blood, 2003, 102, 3075; Damle, R. N., et al., Blood, 1999, 94, 1840-1847; Hamblin, T. J., et al., Blood 1999, 94, 1848-1854; Oscier, D. G., et al., Blood 1997, 89, 4153-4160; Krober, A., et al., Blood, 2002, 100, 1410-1416; and U.S. Patent Application Publication No. 2003/0203416.

The term “subject” as used herein refers to humans, as well as to other primates and to mammals generally.

Protein and nucleic acid sequences for non-human vimentin, annexin 5 and 6-phosphogluconolactonate have been characterized in various species as exemplified in references including Bussemakers M J, Biochem Biophys Res Commun., 182(3):1254-9, 1992; Hess, J. F., et al., Gene 140 (2), 257-259, 1994; Zehner, Z. E., et al., J. Biol. Chem. 262 (17), 8112-8120, 1987; Imai, Y., et al., Eur. J. Biochem. 232 (2), 327-334, 1995; and Sonstegard, T. S., et al., Mamm. Genome 13 (7), 373-379, 2002; and as identified in the National Center for Biotechnology Information database as Accession No. Q8CFX1, mus musculus 6-phosphogluconolactonase; Accession No. NP_{—}037264, rattus norvegicus annexin 5; Accession No. XP_{—}001100224, non-human primate annexin 5; Accession No. NP_{—}001026709, gallus gallus annexin 5; Accession No. AAX09018, bos Taurus annexin 5; Accession No. NP_{—}033803, mus musculus annexin 5; Accession No. P48036, mus musculus annexin 5; UniGene Accession Nos. Hs.480653, ANXA5: Annexin A5 Homo sapiens; Mm.1620, Anxa5: Annexin A5 Mus musculus; Bt.49277, ANXA5: Annexin A5 Bos taurus; Dr.76352 anxa5: Annexin A5 Dania rerio; Rn.3318, Anxa5: Annexin A5 Rattus norvegicus; Spu.14333 LOC763085: Similar to Annexin A5 Strongylocentrotus purpuratus; Gga.1168, ANXA5: Annexin A5 Gallus gallus; Bt.61451, LOC784297: Similar to Annexin A5Bos taurus; Mfa.8271, Macaca fascicularis brain cDNA clone: QbsB-10774, similar to human annexin A5(ANXA5), mRNA, RefSeq: NM_{—}001154.2, Macaca fascicularis, Pta.279, [Pan troglodytes] Pinus taeda; Mus musculus vimentin CAA39807.1; Rattus norvegicus vimentin Accession No. NP_{—}112402.1; and Bos Taurus vimentin Accession No. NP_{—}776394.2.

Antibodies

Antibody compositions are described according to the present invention along with methods including use of the antibodies. In particular, antibodies which bind substantially specifically to a vimentin cleavage fragment, an IMW vimentin cleavage fragment, uncleaved vimentin, annexin 5 and/or 6-phosphogluconolactonase are useful in methods described herein as well as in other applications where specific detection of these antigens is advantageous.

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

In particular embodiments, monoclonal antibodies and methods including use of monoclonal antibodies are provided by the present invention. Monoclonal antibodies are prepared using techniques known in the art such as described in E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003, for example. Monoclonal antibodies according to the present invention and/or used in methods according to the present invention are produced by techniques illustratively including, but not limited to, hybridoma techniques, recombinant nucleic acid methodology and/or isolation from a phage library, for example as described in the above cited references. Monoclonal antibodies are advantageously used in particular embodiments due to the specificity of the binding of monoclonal antibodies which recognize a single epitope.

In certain embodiments, an antibody provided according to the present invention binds substantially specifically to an IMW vimentin cleavage fragment. In further embodiments, a monoclonal antibody provided according to the present invention binds substantially specifically to an IMW vimentin cleavage fragment. In still further embodiments, a monoclonal antibody provided according to the present invention binds substantially specifically to an vimentin cleavage fragment and does not bind substantially specifically to uncleaved vimentin, or cleavage fragments other than IMW vimentin cleavage fragments. For example, a particular monoclonal antibody according to the present invention recognizes an epitope at or near the “cleavage site” of an IMW vimentin cleavage fragment, wherein the epitope is not present and/or not antigenic in uncleaved vimentin and cleavage fragments other than a IMW vimentin cleavage fragment. An IMW vimentin cleavage fragment found to be indicative of a form of chronic lymphocytic leukemia according to particular embodiments of the present invention is cleaved such that the IMW vimentin cleavage fragment includes the N-terminus of uncleaved full-length vimentin but not the standard C-terminus. Thus, the term “cleavage site” in this context refers to the end of an IMW vimentin cleavage fragment at the extremity of the IMW vimentin cleavage fragment distal to the N-terminus. In alternative embodiments, an IMW vimentin cleavage fragment found to be indicative of a form of chronic lymphocytic leukemia according to particular embodiments of the present invention is cleaved such that the IMW vimentin cleavage fragment includes neither the C-terminus of uncleaved full-length vimentin nor the standard N-terminus. In such a case, the term “cleavage site” refers to both termini of the IMW vimentin cleavage fragment and specific monoclonal antibodies are provided which substantially specifically bind to each cleavage site.

Antibodies provided by particular embodiments bind substantially specifically to a specified vimentin cleavage product. For example, a vimentin cleavage product and/or a fragment thereof is used as an immunogen to produce an antibody specific to the vimentin cleavage product. Alternatively, or in combination, full-length vimentin may be used as an immunogen. Exemplary fragments of a vimentin cleavage product illustratively include a peptide at or near the N-terminus and/or C-terminus of the vimentin cleavage product.

Antibodies specific for annexin 5 or 6-phosphogluconolactonase are also provided according to the present invention.

Vimentin, a vimentin cleavage product, annexin 5 or 6-phosphogluconolactonase and/or fragment thereof for use as an immunogen may be obtained by techniques illustratively including isolation from a sample obtained from a subject containing the product or fragment of the product, produced by recombinant techniques or chemically synthesized, for example as described in Harrington, M. G., Methods Enzymol. 182:488-495, 1990; J. Sambrook and D. W. Russell, Molecular Cloning: A Laboratory Manual, Cold Spring Harbor Laboratory Press, 3^{rd} Ed., 2001; and Merrifield, JACS, 85:2149-2154, 1963.

According to one embodiment, a vimentin cleavage product used as an immunogen is an IMW cleavage product. A particular product used as an immunogen is amino acids 2-429 of SEQ ID No. 1, a variant thereof or an immunogenic fragment of SEQ ID No. 1 and/or a variant thereof.

According to one embodiment, a vimentin cleavage product used as an immunogen is an LMW cleavage product. A particular product used as an immunogen is amino acids 86-466 of SEQ ID No. 1, amino acids 86-429 of SEQ ID No. 1, and/or a variant of either.

According to a further embodiment, a particular immunogen is the annexin 5 protein of SEQ ID No. 2, a variant thereof and/or a fragment of either.

According to a further embodiment, a particular immunogen is the 6-phosphogluconolactonase protein of SEQ ID No. 4, a variant thereof and/or a fragment of either.

An immunogenic fragment is a peptide or protein having about 4-500 amino acids, and in particular embodiments, at least 5 amino acids, or in further embodiments, at least 6, 7, 8, 9, 10, 11, 12, 13, 14, 15, 20, 22, 23, 24, 25, 30, 35, 40, 50, 100, 200, 300, or 400 amino acids.

Peptides and/or proteins used as immunogens may be conjugated to a carrier, such as keyhole limpet hemocyanin and bovine serum albumin. An immunogen is administered to an animal in particular methods, such as a rabbit, goat, mouse, rat, sheep or chicken. Immunoglobulins produced in the animal may be obtained from the animal and optionally purified for screening and use.

Optionally, monoclonal antibodies are prepared using techniques known in the art, illustratively including those described in Kohler, G. and Milstein, C., Nature, 256:495-497, 1975; E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

Particular methods of monoclonal antibody preparation include obtaining spleen cells from an animal immunized with an immunogen and fusing the antibody-secreting, lymphocytes with myeloma or transformed cells to obtain a hybridoma cell capable of replicating indefinitely in culture.

A hybridoma cell producing antibodies substantially specific for a marker for a form of CLL is provided according to the present invention.

Antibodies obtained are tested for substantially specific binding to the immunogen by methods illustratively including, ELISA, Western blot and immunocytochemistry.

In addition to an antibody, a binding agent is illustratively an isolated protein or peptide which binds to a molecule of interest with substantial specificity. For example, a binding agent is illustratively an aptamer which substantially specifically binds to a vimentin cleavage fragment, an IMW vimentin cleavage fragment, uncleaved vimentin, annexin 5 and/or 6-phosphogluconolactonase. The term “aptamer” refers to a peptide and/or nucleic acid that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Similarly, peptide-based aptamers are characterized by specific binding to a target wherein the aptamer is not a naturally occurring ligand for the target. Techniques for identification and generation of peptide and nucleic acid aptamers is known in the art as described, for example, in F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3^{rd} Ed., 2001.

Kits for detection of a form of chronic lymphocytic leukemia in a subject are provided according to embodiments of the present invention which include a binding agent for substantially specific binding to a vimentin cleavage product, uncleaved vimentin, annexin 5, and/or 6-phosphogluconolactonase.

In further embodiments of kits according to the present invention, a nucleic acid probe or primer which hybridizes to annexin 5 and/or 6-phosphogluconolactonase RNA under stringent hybridization conditions is included.

Embodiments of particular inventive kits include instructions for use of a binding agent, primer and/or probe in detection of a vimentin cleavage product, uncleaved vimentin, annexin 5, and/or 6-phosphogluconolactonase and detection of a form of chronic lymphocytic leukemia in a subject.

Optionally, a reagent for specifically detecting beta-2-microglobulin, immunoglobulin variable region heavy chain gene, ZAP-70, CD-38, lipoprotein lipase, a chromosomal abnormality associated with chronic lymphocytic leukemia, or a combination of any of these is included in a kit according to the present invention.

Kits provided according to the present invention are useful in diagnosis, prognosis and monitoring of disease, particularly chronic lymphocytic leukemia, in a subject. For example, a kit according to the present invention is useful in ascertaining the form of chronic lymphocytic leukemia in a subject, information which contributes to treatment considerations. In addition, since the form of chronic lymphocytic leukemia may evolve in a subject over the course of the disease, inventive kits are useful in following progress of the disease, such as a change from a less aggressive form of chronic lymphocytic leukemia to a more aggressive form.

Embodiments of kits according to the present invention optionally include one or more components for use in an assay of the present invention illustratively including a liquid such as a buffer and/or solution used in an assay, a container, a detectable label for labeling a binding agent directly or indirectly, a standard, a negative control and a positive control.

Embodiments of inventive compositions and methods are illustrated in the following examples. These examples are provided for illustrative purposes and are not considered limitations on the scope of inventive compositions and methods.

### EXAMPLES

### Example 1

### Samples

Blood samples are obtained from patients who fulfill diagnostic and immunophenotypic criteria for common B-cell CLL, as described in Kipps, T. J., Williams Hematology 2001, 1163-1194, after signing an informed consent form approved by the institutional review boards of the University of California, San Diego. Blood mononuclear cells are isolated by density-gradient centrifugation over Ficoll-Hypaque (Pharmacia, Uppsala, Sweden). Cells are suspended in freezing medium containing 20% fetal bovine serum (FBS) and 5% DMSO for storage in liquid nitrogen. The mutational and ZAP-70 status is determined as described in Rassenti, L. Z., Huynh, L., Toy, T. L., Chen, L., et al., N Engl J Med 2004, 351, 893-901 and shown in Table 1.

Table 1 indicates patients in this study and their respective mutational and ZAP-70 status. CLL samples included in this study are marked with the corresponding patient ID number. For each CLL sample ZAP-70 levels (% ZAP) and percent sequence homology to the corresponding germ-line IgV_{H} is determined (% VH). Sequences with less then 98% VH are considered mutated (mutational status +) (M-CLL). Sequences with 98% VH or more are considered unmutated (mutational status −) (UM-CLL). % ZAP above 20 percent are considered positive (ZAP positivity +). % ZAP below 20 percent is considered negative (ZAP positivity −). The CLL sample from CLL#1 is discordant for its ZAP-70 expression. Patient CLL#8 and CLL#9 are borderline defining their mutational status as described in more detail below.

| | | mutational | | | ZAP |
|---|---|---|---|---|---|
| Patient ID | | status | % VH | % ZAP | positivity |
| | CLL#1 | − | 100 | 14.3 | − |
| | CLL#2 | − | 100 | 67.1 | + |
| | CLL#3 | − | 100 | 43.2 | + |
| | CLL#4 | − | 100 | 63.3 | + |
| a) | CLL#5 | − | 100 | 31.2 | + |
| | CLL#6 | − | 100 | 36.5 | + |
| | CLL#7 | − | 99.6 | 87.1 | + |
| | CLL#8 | − | 98 | 44.4 | + |
| | CLL#9 | + | 96.1 | 10.1 | − |
| a) | CLL#10 | + | 95.4 | 5.2 | − |
| | CLL#11 | + | 94.7 | 6.2 | − |
| | CLL#12 | + | 94.3 | 0.4 | − |
| | CLL#13 | + | 90.1 | 0.4 | − |
| | CLL#14 | + | 90 | 0.3 | − |

(a) Two samples of this patient were taken with six months interval and separately added to the analysis to evaluate the reproducibility of the method and protein expression (even over long time-interval).

Sequences with less then 98 percent homology to the corresponding germ-line IgV_{H} sequence are considered mutated as described in Rassenti et al., supra. ZAP-70 levels above 20 percent are considered positive as described in Rassenti, et al., supra.

Flow cytometry analysis is performed to analyze the purity of the B-CLL samples. The following conjugated antibodies are purchased from BD PharMingen (San Diego, Calif.): fluorescein isothiocyanate (FITC)-labeled anti-CD19, R-phycoerythrin (R-PE)-labeled anti-CD5, peridinin chlorophyll protein (PerCP)-labeled anti-CD3. Stained cells are analyzed on a FC500P™ (Beckman Coulter, Nyon, Switzerland) flow cytometer and flow cytometry data are analyzed using Cytomixs RXP software (Beckman Coulter, Nyon, Switzerland). This phenotypic analysis showed that all CLL-samples in this analysis comprised more than 97% of double positive CD5+/CD19+ cells and less than 3% of CD3+ cells. Blood samples of 38 CLL patients (19 UM-CLL (ZAP-70 positive) and 19 M-CLL (ZAP-70 negative)), are used to confirm the results by Western blot analysis.

### Example 2

### Protein Extraction

Protein extraction is performed using the Ready Prep sequential extraction Kit from Biorad (Biorad, Hercules, Calif., USA). Frozen CLL blood mononuclear cell samples are thawed gently and washed 3 times at 4° C. in cool PBS (Gibco, Invitrogen Inc.). Pellets are resuspended in buffer I of the ReadyPrep Sequential Extraction Kit (Biorad) containing 40 mM Trisbase. To protect the samples from phosphatase and protease activity a cocktail of phosphatase inhibitors (Sigma, Steinheim, Germany) containing: cantharidin, bromotetramisole, microcystin LR, sodium orthovanadate, sodium molybdate, sodium tartrate and imidazole) and broad range protease inhibitors, inhibiting serine, cysteine and metalloproteases as well as calpains (Roche diagnostics, Mannheim, Germany) is added to each sample. Nucleic acids, which can disturb isoelectrical focusing and gel electrophoresis, as described in Shaw, M. M. and Riederer, B. M., Proteomics 2003, 3, 1408-1417, are removed from the samples by treatment with Benzonase (endonuclease) (Sigma, Steinheim, Germany). Cells are lysed by sonication for 10 minutes in a 35 kHz sonication bath (Retsch, Germany). After centrifugation, precipitating the non-soluble proteins, the protein concentrations of the soluble proteins present are measured using the Coomassie Protean Reagent assay from Pierce (Pierce, Rockford, USA) according to the guidelines provided by the manufacturer.

### Example 3

### Two-Dimensional Gel Electrophoresis

Fifty micrograms of the soluble protein extract is precipitated with cold acetone overnight at −20° C. After centrifugation at 20000 g for 5 minutes, the pellet is air dried. Two-dimensional gel electrophoresis (2-DE) is performed according to Görg et al. [Görg, A., Obermaier, C., Boguth, G., Harder, A., et al., Electrophoresis 2000, 21, 1037-1053] with minor adjustments. Briefly, 50 micrograms of the soluble protein extract of the CLL cells of each patient is dissolved in 340 microliters of rehydration buffer containing 7 M urea, 2 M thiourea, 4% (w/v) CHAPS, 20 mM DTT and 0.2% (w/v) Carrier Ampholyte solution (Amersham Biosciences, Uppsala, Sweden). Once the pellet is completely dissolved, each sample is incorporated overnight by rehydration in a 17 cm linear IPG strip (Biorad, Hercules, Calif., USA) with a pH gradient of 3 up to 10. After in-gel rehydration, the strips are iso-electrically focused on the protean IEF cell (Biorad, Hercules, Calif., USA) at 18° C., using 250 V for 60 min (linear ramping), 500 V for 1 h (linear ramping), 1 kV for 2 h (linear ramping), rapid ramping to 10 kV in 3.5 h and steady state at 10 kV for 48 kVh. After iso-electric focusing, the strips are equilibrated in two steps by gentle shaking for 15 minutes in a solution containing TrisHCl buffer (50 mM, pH 8.8), 6M urea, 20% (w/v) glycerol and 2% (w/v) SDS. Proteins are reduced and alkylated by adding 1.5% (w/v) DTT to the first, and 4% (w/v) iodoacetamide to the second step. After equilibration, the IPG strips are placed on a polyacrylamide gel (12% T; 2.6% C) and run in sets of 6 in the vertical Protean II xi Multi cell (Biorad, Hercules, Calif., USA) at 16 mA/gel for 30 minutes and 32 mA/gel for approximately 4 hours and 30 minutes at 10° C. The 18×20 cm 2-DE gels are stained using silver staining according to Stults and O'Connell as described in O'Connell, K. L. and Stults, J. T., Electrophoresis 1997, 18, 349-359.

Preparative gels are run in order to identify less abundant proteins. An average of 300 micrograms of soluble proteins is loaded on these preparative gels and they are stained using Sypro Ruby (Biorad, Hercules, Calif., USA) according to the manufacturer's guidelines. In terms of sensitivity, Sypro Ruby is largely similar or even more sensitive than routinely used silver staining methods as described in White, I. R., Pickford, R., Wood, J., Skehel, J. M., et al., Electrophoresis 2004, 25, 3048-3054; Patton, W. F., Electrophoresis 2000, 21, 1123-1144, but offering a higher level of compatibility with the identification of proteins by mass spectrometry as detailed by White, I. R., et al., Electrophoresis 2000, 21, 1123-1144.

### Example 4

### 2-DE Gel Imaging and Data Analysis

Silver stained 2-DE gels are scanned and digitized using a GS-800 Densitometer from Biorad (Biorad, Hercules, Calif., USA). Digitized images are analyzed with PDQuest 2D-analyser Software v 7.1 (Biorad, Hercules, Calif., USA). Spots are detected using the spot detection wizard function of the software. Background and both horizontal and vertical streaks are removed by applying the background subtraction tool in the PDQuest Software. In order to compare protein expression levels across the gels in this analysis non-expression-related variations in spot intensity are compensated for by normalizing the protein expression data of each gel for its total density in the gel image. Statistical analysis of the data is accomplished using the PDQuest software.

Statistical analysis of the data is performed according to Aliya et al., described in Alaiya, A. A., Franzen, B., Hagman, A., Dysvik, B., et al., Int J Cancer 2002, 98, 895-899 and Tan et al., described in Tan, F. L., Moravec, C. S., Li, J., Apperson-Hansen, C., et al., Proc Natl Acad Sci USA 2002, 99, 11387-11392, by using the SPSS software (SPSS, Chicago, Ill., USA). Test for normality and equal variances are performed in order to examine whether the data qualified for parametric (Student's T-test) or non-parametric statistical tests (Mann-Withney U-test).

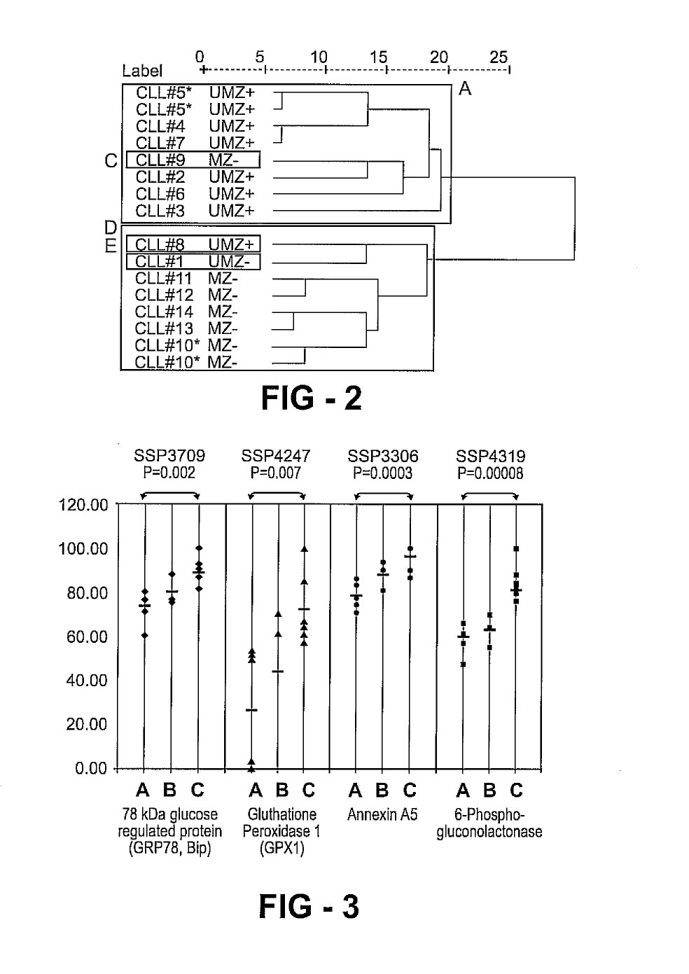

The majority of the data is not normally distributed (Shapiro-Wilk P<0.05) with equal variances (equal variances test, P>0.05). However, some protein expression levels showed a normal distribution (Shapiro-Wilk P>0.05). Therefore, according to methods described in Tilleman et al., detailed in Tilleman, K., Van Beneden, K., Dhondt, A., Hoffman, I., et al., Proteomics 2005, 5, 2247-2257, those proteins that are significantly differentially expressed according to the Mann Whitney Test (p<0.05) and the Student's T-test (p<0.05, after Log-transformation) are investigated using the PDQuest software. The corresponding data are used as variables to perform hierarchical clustering analysis utilizing the SPSS v.11.5 software (SPSS inc. Chicago, Ill., USA). The between group linkage method and squared Euclidian distance similarity measure for clustering analysis is used. The result of the cluster analysis is displayed in a dendrogram, shown in FIG. 2.

The dendrogram in FIG. 2 shows patterns obtained from different patients which are marked designating patient number, mutational status where M indicates mutated and UM indicates unmutated, as well as ZAP-70 status where Z− indicates ZAP-70 negative and Z+ indicates ZAP-70 positive. Expression patterns from patients with no mutations in the IgV_{H} and ZAP-70 positive (UMZ+) cluster in A. Patterns from patients with the mutations in the IgV_{H} and negative for the expression of ZAP-70 (MZ−) cluster in B. Protein expression patterns from patient CLL#8 and CLL#9 did not cluster correctly according their mutational or ZAP-70 status, respectively shown at D and C. The pattern from the ZAP-70 discordant sample in the unmutated (UMZ) group clusters with patterns obtained from patients with the same ZAP-70 status, shown at E.

### Example 5

### Protein Identification by LC-MS/MS Analysis

Spots of interest are excised from the preparative gels, destained and digested with sequencing grade modified trypsin (Promega) as described by Tilleman et al. in Tilleman, K., Van Beneden, K., Dhondt, A., Hoffman, I., et al., Proteomics 2005, 5, 2247-2257. The resulting peptide mixture is analyzed using an automated nanoscale liquid chromatography (nano-LC) system and tandem mass spectrometry (MS/MS) detailed in Devreese, B., Vanrobaeys, F. and Van Beeumen, J., Rapid Commun Mass Spectrom 2001, 15, 50-56. The Ultimate capillary LC system equipped with a FAMOS autosampler and Switchos column switching system (LC Packings, Sunnyvale, Calif., USA) coupled to a hybrid quadrupole orthogonal acceleration time-of-flight tandem mass spectrometer (Q-TOF; Micromass, Manchester UK) fitted with an orthogonal nano Z-spray, detailed in Vanhoutte, K., Van Dongen, W., Hoes, I., Lemiere F., et al., Anal Chem 1997, 69, 3161-3168, is used.

Briefly, peptides are concentrated and desalted on a reversed-phase (RP) PepMap C18 precolumn (Micro Guard Column) (300 micron ID, 1 mm length; LC Packings) with a flow of 10 microliters/min. Further, the flow direction over the precolumn is switched and gradient elution using a flow rate of 150 nl/min transfers the peptides to the RP capillary PepMap C18 column (3 microns, 100 Ångstroms, 75 microns ID, 15 cm length; LC Packings) where the peptide separation is performed prior to online electrospray ionization (ESI) and tandem mass spectrometric analysis (MS/MS). The ions of interest are selected for MS/MS collision induced fragmentation (CID) by the Automatic Function Switching option in the masslynx software (Waters, Manchester, UK) as detailed in Devreese, B., Vanrobaeys, F. and Van Beeumen, J., Rapid Commun Mass Spectrom 2001, 15, 50-56; Hoyes, E. and Gaskell, S. J., Rapid Commun Mass Spectrom 2001, 15, 1802-1806. Proteins are identified using the Proteinlynx global server bio-informatics platform (v. 2.0) (Waters, Manchester, UK) to process the mass spectrometric data and to screen the Swiss-Prot database in combination with the MS/MS ion search function of Mascot (Matrix Science Ltd.).

### Example 6

### Western Blot Analysis

Prior to immunodetection, proteins are separated either by 1-dimensional (1-DE) or 2-DE denaturing sodium dodecyl sulphate-polyacrylamide gel electrophoresis (SDS-PAGE) on a 10% (12% for annexin) polyacrylamide gel. For 1-DE SDS-PAGE, 10 micrograms of the soluble (acetone precipitated) protein extract is dissolved in sample loading buffer (1% SDS, 10% glycerol, 10 mM Tris-HCl (pH 6.2) 1 mM EDTA) with 5% beta-mercaptoethanol (v/v) and heated for 5 min at 95° C. prior to direct loading on the SDS-PAGE separation gel. For 2-DE SDS-PAGE sample preparation and isoelectrical focusing is performed as described above using a 11 cm linear IPG strip (Biorad, Hercules, Calif., USA) with a pH gradient of 4 up to 7. Electrophoresis is performed using 200 V (constant voltage). The proteins separated by 1-DE or 2-DE are transferred onto a nitrocellulose membrane (Trans-blot Transfer medium; Biorad) using a tank Trans-blot Electrophoretic Transfer Cell system (Biorad). Prior to electrophoretic transfer, the gels and nitrocellulose membranes are pre-equilibrated in transfer buffer (containing 48 mM Tris, 39 mM Glycine and 20% Methanol) which is further used to fill the buffer system. Electrophoretic transfer is performed at 50 V (constant voltage) for 5 hours. Protein transfer and equal loading is evaluated by Ponceau staining. Prior to immunodetection, blots are blocked in 10 mM TBS buffer containing 0.1 (v/v) Tween-20 (TBS-T) 5% non-fat dry milk (Biorad) at room temperature with gentle shaking for 1 h. Blots are incubated overnight in plastic bags with the appropriate primary antibody dilution on a rotary shaker at 4° C. The monoclonal mouse anti-vimentin antibody (Clone V9, Sigma-Aldrich, Inc.), anti-annexin V (clone 130, Santa-Cruz biotechnology, Inc.) and the goat polyclonal antibody against L-plastin ((D-16), Santa-Cruz biotechnology, Inc.) diluted respectively 1/400, 1/500 and 1/100 in TBS-T (0.1% milk) are used in this example. After 3 washing steps with TBS-T (0.1% milk) for 5 min, blots are incubated with the appropriate horseradish peroxidase-conjugated polyclonal antibody diluted in TBS-T (0.1% milk) for 1 h at room temperature. Finally, blots are washed and the chemiluminescence substrate (SuperSignal West Dura Extended Duration substrate, Pierce) is added according to the manufacturer's recommendations. Digitized images are acquired using a Versadoc™3000 imaging system (Biorad).

### Example 7

### 2-DE Results and Analysis

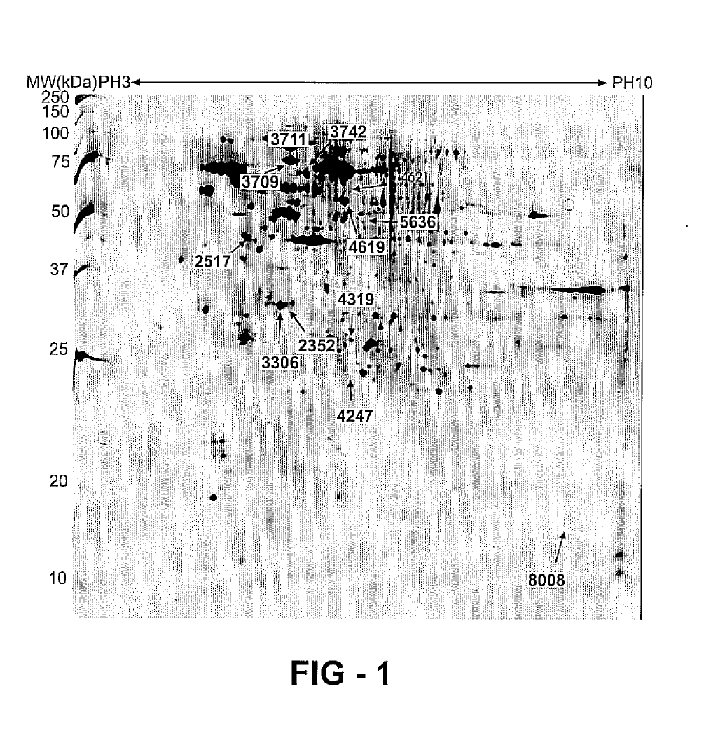

In an initial analysis, 7 UM-CLL samples and 5 M-CLL samples are analyzed. To extend and confirm the initial analysis, the reproducibility of the method and the reproducibility of protein expression in samples from subjects, even over a long time-interval, additional samples are added, see below. The UM-CLL group contains 1 patient who is discordant for ZAP-70 expression designated CLL#1 UMZ−, see Table 1. Reproducible gel-images are obtained and an average total number of 991 spots are resolved on each gel, a representative digitized image of which is shown in FIG. 1.

FIG. 1 shows a representative 2-DE gel image from a gel run as described in Example 3. The pH range was 3-10 and the molecular weight standard shows the separation in the second dimension from 250 to 10 kDa. Protein spots marked with their SSP number (given by the PDQuest software) are found to be differentially expressed and are listed in Table 2.

In a first statistical analysis, proteins significantly differentially expressed P<0,05) between the M-CLL and UM-CEL patients are selected. In a second analysis the ZAP-70 discordant sample is omitted from the statistical analysis. These analyses result in several datasets. However, clustering analyses using any of these datasets indicated aberrant protein expression in 2 patients, designated CLL#8 UMZ+ and CLL#9 MZ−, compared to other members with the same mutational or ZAP-70 expression. Strikingly, these two patients who did not join the expected cluster are around the cut off for determining their mutational status, patient CLL#8 UMZ+ shows 98% and CLL#9 MZ− 96,1% homology to the corresponding germ-line IgV_{H} sequence, shown in Table 1. As the conventional cut off of 98% sequence identity to the nearest germ line IgV_{H} sequence is used in this analysis, we annotated patient CLL#8 and CLL#9 respectively as UM-CLL and M-CLL. However, the optimal cut off for this distinction is not clear, Wiestner, A., Rosenwald, A., Barry, T. S., Wright, G., et al., Blood 2003, 101, 4944-4951; Davis, Z. A., Orchard, J. A., Corcoran, M. M. and Oscier, D. G., Blood 2003, 102, 3075. Early studies used a cut off of 98% sequence identity to allow for germ line immunoglobulin polymorphisms in the human population, Damle, R. N., Wasil, T., Fais, F., Ghiotto, F., et al., Blood 1999, 94, 1840-1847; Hamblin, T. J., Davis, Z., Gardiner, A., Oscier, D. G. and Stevenson, F. K., Blood 1999, 94, 1848-1854; Oscier, D. G., Thompsett, A., Zhu, D. and Stevenson, F. K., Blood 1997, 89, 4153-4160. In a recent study with 300 patients, a cut off of 97% sequence identity was optimal for distinguishing patients with CLL that had different overall survival rates, Krober, A., Seiler, T., Benner, A., Bullinger, L., et al., Blood 2002, 100, 1410-1416. However, the 95% confidence interval for this distinction ranged from 96% to 98% sequence identity, Wiestner, A., Rosenwald, A., Barry, T. S., Wright, G., et al., Blood 2003, 101, 4944-4951. This might indicate that patient CLL#8 and CLL#9 could potentially have been mistakenly annotated in their IgV_{H} mutational status. Therefore the data are reanalyzed excluding these two samples and the ZAP-70 discordant sample, CLL#1 UMZ−, and this is to be considered the training set for the cluster analysis. This statistical analysis results in 23 spots which are differentially expressed (p<0.05) between CLL-cells from the remaining 5 M-CLL patients who are ZAP-70 negative (MZ−) and the remaining 6 UM-CLL patients who are ZAP-70 positive UMZ+). The identified proteins, obtained from these 23 spots are shown in Table 2. Most of these spots show a higher expression in CLL cells from UM-CLL patients.

TABLE 2

| | | | | | | | Students | Micro-array | |
|---|---|---|---|---|---|---|---|---|---|
| | | | | | Average | Average | T-Test | data {*} | |
| SSP | AN | Protein name | | Function | M | UM | P | P | |
| 2352 | P08758 | Annexin AS | | Phospholipid | 45 | 295 | 0.0214 | — | |
| | | | | Binding | | | | | |
| 2517 | P08670 | Vimentin | | Intermediate | 41 | 390 | 0.0084 | 0.1317 | c) |
| | | | | Filament | | | | | |
| 3306 | P08758 | Annexin AS | | | 894 | 3296 | 0.0003 | — | |
| 3709 | P11021 | 78 kDa glucose regulated | a) | ER resident | 575 | 2086 | 0.0024 | 0.0007 | b) |
| | | protein (GRP78, Bip) | | Protein | | | | | |
| 3742 | P13796 | L-plastin (LCP-1) | | Actin-binding | 76 | 1482 | 0.0215 | 0.002 | b) |
| 4247 | P07203 | Glutathione peroxidase 1 | | Protects from oxidative | 23 | 386 | 0.0074 | 0.0005 | b) |
| | | (GPX1) | | Breakdown | | | | | |
| 4319 | O95336 | 6-phosphogluconolactonase | a) | Metabolism | 122 | 663 | 0.0007 | — | |
| | | (6PLG) | | | | | | | |
| 4619 | P30101 | Protein disulfide isomerase | a) | ER resident protein | 488 | 2616 | 0.0045 | 0.0012 | b) |
| | | A3 (Erp57) | | | | | | | |
| 4621 | P17987 | T-complex protein 1, alpha | a) | Molecular chaperone | 18 | 116 | 0.0191 | — | |
| 5636 | P18031 | Protein tyrosine phosphatase | | Signal transduction | 3 | 82 | 0.0015 | 0.18 | c) |
| | | 1B | | | | | | | |
| 3711 | P11142 | Heat shock cognate 71kDa | a) | Chaperone | 313 | 27 | 0.0004 | — | |
| | | protein (HSC70) | | | | | | | |
| 8008 | P07737 | Profilin I | | Binds to actin, PIP2 | 113 | 4 | 0.00003 | 0.005 | c) |

[*] Wiestner, A., Rosenwald, A., Barry, T. S., Wright, G., et al., Blood 2003, 101, 4944-4951

To evaluate the results of the analysis, the reproducibility of the method and the protein expression in CLL samples, even over a long time-interval, additional samples are added to the analysis. Further, in two patients, CLL#5 and CLL#10, a first sample is obtained and a second sample is obtained six months later, see Table 1. These samples are added separately to the analysis, as a validation test. Including these samples in the statistical analysis did not change the significance (P<0.05) of the difference in expression level of the 23 protein spots described above.

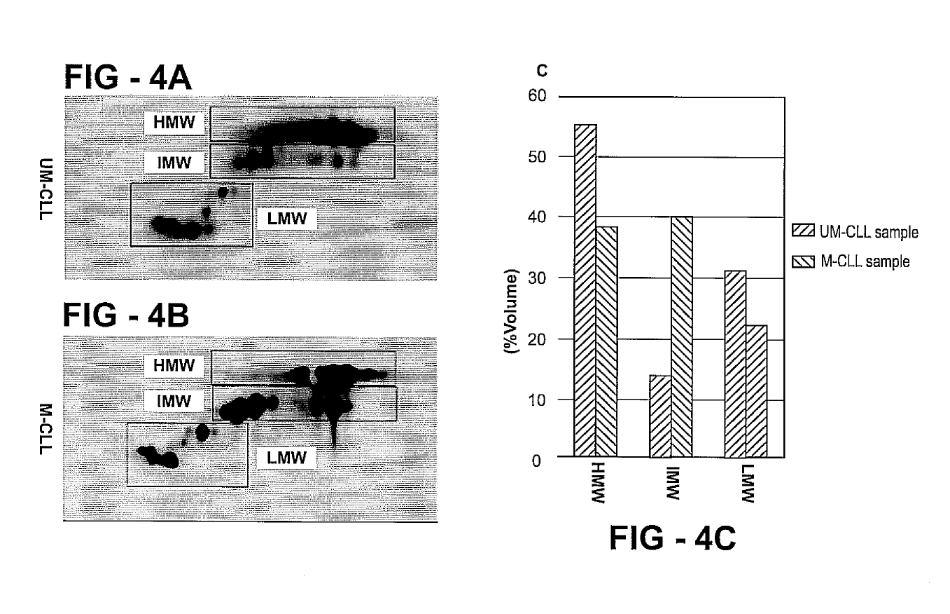

FIG. 3 shows the relationship between the relative normalized expression levels (y-axis) of a particular protein in each CLL sample and the CLL subgroup these samples were assigned to α-axis) for several spots resulting from the statistical analysis. This figure shows intermediate protein expression level in patient CLL#8, CLL#9 and the ZAP-70 discordant sample, CLL#1 UMZ−, compared to the more homogenous expression level in the remaining ZAP-70 concordant samples with the same mutational status. The M-CLL (A) and UM-CLL (C) group contained respectively 6 and 7 CLL samples, duplicate gels of CLL#5 and CLL#10 included. Patient CLL#8, CLL#9 and the ZAP-70 discordant sample CLL#1 are represented as a separate subgroup (B). Each dot corresponds to the relative spot intensity in an individual gel relative to the highest expression level for the particular spot in the analysis. Average expression levels in each group are marked with a “-”. Spots included in this figure are marked with their SSP number (given by the PDQuest software) and their protein name, respectively above and below the corresponding part of the figure. P values were calculated using the Student's T-test on the log transformed data.

### Example 8

### Classification by Hierarchical Clustering Analysis

A subset of spots is used in a clustering analysis to examine whether the expression level of these proteins can be used to stratify patients according their IgV_{H} mutational and/or ZAP-70 status. A subset of spots derived from the differential analysis of the training set as described above is used as described in Helmann, K., Alaiya, A. A., Schedvins, K., Steinberg, W., et al., British Journal of Cancer 2004, 1-8. The protein expression patterns of CLL#8 and CLL#9 did not cluster according their ZAP-70 or mutational status. The protein expression patterns of the remaining patients concordant for mutational status and ZAP-70 expression nicely clustered together. The duplicate samples from patient CLL#5* (and CLL#10* from the validation test, taken with a six months interval and which are not included in the training set, cluster together in the correct cluster, showing that the protein expression between these duplicate samples is more similar within the sample duplicates than with any other sample. This demonstrates the reproducibility of the method and the stability of the protein expression, over a 6 months time-interval. The protein expression pattern of the ZAP-70 discordant sample, CLL#1 UMZ−, clustered with those obtained from patients with the same ZAP-70 status.

### Example 9

### Protein Identification

Protein spots that are significantly differentially expressed in the statistical analyses are selected for identification. Two of the proteins (protein disulfide isomerase (ERp57, also known as PDIA3, ER-60, GRP58, ERp60), Profilin I) are also identified by Voss et al., in Voss, T., Ahorn, H., Haberl, P., Döhner, H. and Wilgenbus, K., International Journal of Cancel 2001, 91, 180-186, to be differentially expressed between patients in correlation to survival. None of them are described by Cochran et al., in Cochran, D. A. E., Evans, C. A., Blinco, D., Burthem, J., et al., Molecular & Cellular Proteomics 2003, 2, 1331-1341, to be differentially expressed between patients in correlation to the mutational status of the IgV_{H} region. These analyses used different experimental conditions, such as a smaller pH range and different statistical methods. None of them included the ZAP-70 status in the analysis.

The presently reported analysis, using a pH range 3-10 and including the ZAP-70 status results in the identification of several interesting proteins, including vimentin, annexin A5,6-phosphogluconolactonate and several others, as shown in Table 2.

Protein expression of the identified proteins is compared to the publicly available data from DNA microarray analysis comparing mRNA expression in the different CLL subtypes, Wiestner, A., Rosenwald, A., Barry, T. S., Wright, G., et al., Blood 2003, 101, 4944-4951; Klein, U., Tu, Y., Stolovitzky, G. A., Mattioli, M., et al., J Exp Med 2001, 194, 1625-1638; Rosenwald, A., Alizadeh, A. A., Widhopf, G., Simon, R., et al., J Exp Med 2001, 194, 1639-1647; Haslinger, C., Schweifer, N., Stilgenbauer, S., Dohner, H., et alt, J Clin Oncol 2004, 22, 3937-3949; Ferrer, A., Ollila, J., Tobin, G., Nagy, B., et al., Cancer Genet Cytogenet 2004, 153, 69-72. Primary data from Rosenwald et al., in Rosenwald, A., Alizadeh, A. A., Widhopf, G., Simon, R., et al., J Exp Med 2001, 194, 1639-1647 and Wiestner et al., in Wiestner, A., Rosenwald, A., Barry, T. S., Wright, G., et al., Blood 2003, 101, 4944-4951, are downloaded from the website http://Ilmpp.nih.gov/cll/. Data from Klein et al., Klein, U., Tu, Y., Stolovitzky, G. A., Mattioli, M., et al., J Exp Med 2001, 194, 1625-1638, are downloaded from the supplemental tables available at http://www.jem.org/cgi/content/full/194/11/1625/.

Statistical analysis is performed on these data as described in Wiestner, A., Rosenwald, A., Barry, T. S., Wright, G., et al., Blood 2003, 101, 4944-4951 who used a (2-group t statistic) Student's T-test on log-transformed mRNA expression ratios to measure the ability of each array element to discriminate between the 2 IgV_{H} mutation subtypes. No primary data are available from Haslinger, C., Schweifer, N., Stilgenbauer, S., Dohner, H., et al., J Clin Oncol 2004, 22, 3937-3949 or Ferrer, A., Ollila, J., Tobin, G., Nagy, B., et al., Cancer Genet Cytogenet 2004, 153, 69-72, but the proteins found in the present study are compared to the results published.

mRNA expression data are available for 7 proteins resulting from our proteomic analyses. The results obtained from Wiestner, A., Rosenwald, A., Barry, T. S., Wright, G., et al., Blood 2003, 101, 4944-4951 are included in Table 2 and positive correlation of those results with the present protein expression data is marked b). It is noted that mRNA expression data found in Rosenwald, A., Alizadeh, A. A., Widhopf, G., Simon, R., et al., J Exp Med 2001, 194, 1639-1647 are also found in Wiestner, A., Rosenwald, A., Barry, T. S., Wright, G., et al., Blood 2003, 101, 4944-4951.

Interestingly, a negative correlation with mRNA expression results of Wiestner et al., supra, and Rosenwald et al., supra, marked c) in Table 2 is observed for some proteins, particularly vimentin. This is important since differences in protein expression can be caused by different post-translational modifications in addition to, or instead of differences due to mRNA expression. This emphasizes the need for performing proteome analysis even if microarray data are available. Therefore validation of proteome results obtained in the present examples and described herein by performing quantitative PCR analysis does not necessarily reveal information advantageously obtained by performing protein analysis, such as Western blotting. In addition, it is noted that several proteins, particularly annexin 5 and 6-phosphogluconolactase, identified as differentially expressed in CLL patients were not apparently reported previously as differentially expressed by Wiestner et al., supra, or Rosenwald et al., supra.

### Example 10

### Immunodetection

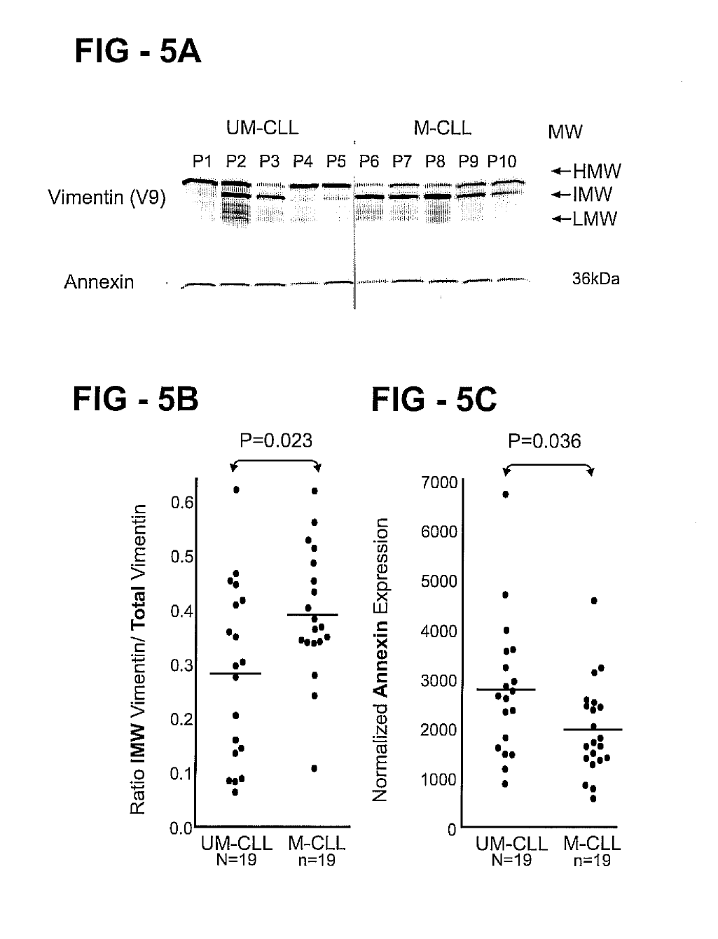

One of the proteins found to be differentially expressed between UM-CLL and M-CLL is identified as a vimentin protein fragment. To evaluate differential vimentin processing in more detail, Western blot analysis is performed after 2-DE gel electrophoresis of the soluble proteins obtained from two CLL samples with different mutational status, shown in FIG. 4A, corresponding to a UM-CLL patient sample and FIG. 4B corresponding to a M-CLL patient sample.

Vimentin forms classified according to differences in molecular weight, namely high molecular weight HMW), intermediate molecular weight (IMW) and low molecular weight (LMW) vimentin forms are identified, see FIGS. 4A and 4B. The total signal intensity inside a defined boundary (volume) is calculated by image analysis software (Biorad) and expressed as a percentage of all volumes (% volume) in the image, FIG. 4C. FIG. 4C shows the % volume for the 3 regions, HMW, IMW and LMW, on the 2-DE western blot of vimentin in the UM-CLL and M-CLL samples. These results show the higher abundance of LMW and HMW vimentin in the UM-CLL sample. The IMW vimentin fragments show higher abundance in the M-CLL subgroup.

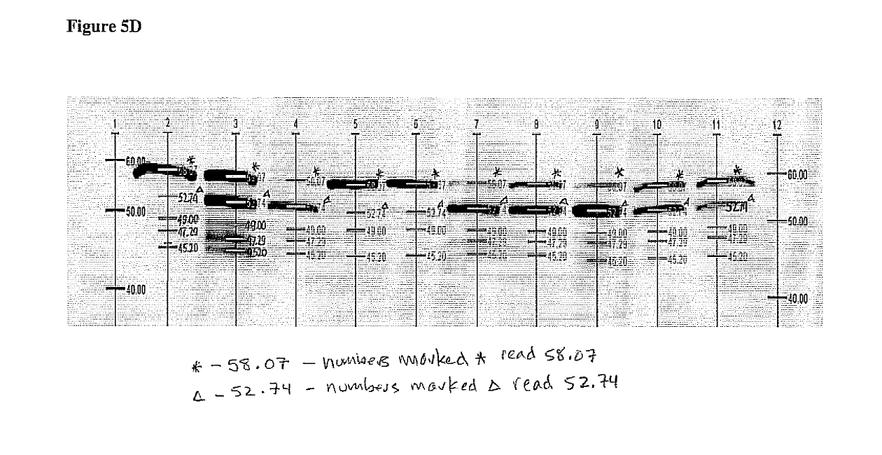

An expression ratio of vimentin forms is calculated relative to the total vimentin expression in each sample in this example. The UM-CLL sample shows a higher abundance of the HMW and LMW vimentin forms, whereas the (IMW) vimentin is most abundant in the M-CLL sample. The relative expression of the intermediate molecular weight (IMW) vimentin fragments is found the most discriminating between the UM-CLL and M-CLL samples. Differential vimentin expression is evaluated by Western blot analysis in 38 CLL samples (19 M-CLL (ZAP-70 negative), 19 UM-CLL (ZAP-70 positive)) after 1-DE, shown in FIG. 5A. Relative expression of the IMW vimentin fragments is confirmed to be significantly lower (P=0.02) in the UM-CLL samples.

Annexin expression is also evaluated by 1-DE Western blot analysis. FIG. 5A is representative for the complete Western blot analysis (19 M-CLL (ZAP-70 negative) and 19 UM-CLL (ZAP-70 positive)) and shows that annexin is expressed in all CLL samples. The normalized annexin expression values confirm the higher expression of annexin in the UM-CLL samples. In addition, 2-DE Western blot analysis is used to confirm the identification of L-plastin and the two different annexin spots (SSP 2352 and SSP 3306) in the UM-CLL group.

FIG. 5 shows representative results of the Western blot analysis in 38 CLL samples including 19 UM-CLL, ZAP-70 positive and 19 M-CLL, ZAP-70 negative samples. Samples labeled P1-P5 in FIG. 5A are obtained from CLL patients with no somatic mutations in the IgV_{H}. Samples labeled P6-P10 in FIG. 5A are obtained from CLL patients with somatic mutations in the IgV_{H}. Differential vimentin and annexin expression between UM-CLL and M-CLL in shown in FIG. 5A. Vimentin fragments defined as either high molecular weight (HMW), intermediate molecular weight (IMW) or low molecular weight (LMW) vimentin forms are indicated. In addition, annexin 5 expression is detected as illustrated in FIG. 5A. FIG. 5B shows the expression ratio of IMW vimentin/total vimentin in all samples according to their mutational status. FIG. 5C shows the normalized expression ratio of annexin A5 in all samples according to their mutational status. P values indicated in FIGS. 5B and 5C are derived from the Student's t-test on the Log transformed expression data and show significant difference.

FIG. 5D shows an image of the gel shown in FIG. 5A with superimposed molecular weights. Approximate Molecular Weight of Vimentin fragments are determined using Magic Mark®, commercially available from Invitrogen, Carlsbad, Calif. The molecular weight standard is shown in lanes 1 and 12. The molecular weight of the HMW fragment is approximately 58 kDa, the molecular weight of the IMW fragment is approximately 53 kDa and the molecular weight of the LMW fragments is equal or lower than approximately 49 kDa. In FIG. 5D image lane 2 corresponds to lane P1 in FIG. 5A, lane 3 corresponds to lane P2, lane 4 corresponds to lane P3, lane 5 corresponds to lane P4, lane 6 corresponds to lane P8, lane 7 corresponds to lane P6, lane 8 corresponds to lane P7, lane 9 corresponds to lane P8, lane 10 corresponds to lane P9, and lane 11 corresponds to lane P10.

### Example 11

The size of molecular weight (LMW) human vimentin cleavage products is assigned based on experimental observations. In particular, it is observed that LMW cleavage products appear to lack the vimentin ‘head’ of human vimentin, amino acids 2-95.

The low molecular weight fragment determined in the proteomics analysis was identified by three different peptides, ILLAELEQLK, LGDLYEEEMR and MALDIEIATYR amino acids 130-139, 146-155 and 391-401, respectively, using mass spectrometry. None of these identified peptides obtained from the low molecular weight vimentin matched sequences present in the vimentin head region.

The total molecular weight of the vimentin ‘head’ region when cleaved at Asp85 (AZ 2-85) is 8724 kDa which corresponds to the molecular weight difference between HMW vimentin and the ‘largest’ LMW vimentin forms (approximately 49 kDa). Additional cleavage at Asp 429 results in fragments of approximately 45 kDa.

Further, an anti-vimentin antibody, monoclonal antibody clone V9, directed against the vimentin tail region, around amino acid 417 binds the LMW cleavage products.

Prediction of the isoelectric point for the various cleavage products is calculated using any of various standard methods and tools, such as the “compute MW/PI” tool on the Expasy proteomics server at http://www.expasy.org.

For the complete full length amino acid sequence of vimentin, 466 amino acids, a theoretical pI of 5.06 is calculated. For a cleavage fragment from position 2 to position 429, a theoretical pI of 5.04 is calculated. For a cleavage fragment including amino acids 85 to 466, a theoretical pI of 4.73 is calculated. The reduced pI of vimentin cleavage fragments lacking the vimentin head explains the acidic shift of the LMW vimentin cleavage fragments observed in FIG. 4. The lower amount of spots with the same molecular weight, but different pI, results from the reduced amount of phosphorylation sites after losing the vimentin ‘head’ sequence. However, some phosphorylation sites at the tail sequence can be phosphorylated as well.

In contrast, human IMW vimentin cleavage fragments only lack a part of the vimentin ‘tail’, and likely are products of cleavage at Asp 429. Thus, for example, the total molecular weight of the vimentin tail cleaved at Asp429, that is amino acids 429-466, is about 4209 kDa which corresponds to the calculated molecular weight difference between HMW and IMW vimentin in Western blot analysis, above. Further, an anti-vimentin antibody, monoclonal antibody clone V9, directed against the vimentin tail region, around amino acid 417 binds the IMW cleavage product.

### Example 12

Exemplary protein and protein encoding sequences detected according to methods of the present invention are shown in this example.

```
SEQ ID No. 1 Vimentin
    1 mstrsvssss yrrmfggpgt asrpsssrsy vttstrtysl gsalrpstsr slyasspggv
  61 yatrssavrl rssvpgvrll qdsvdfslad aintefkntr tnekvelqal ndrfanyidk
121 vrfleqqnki llaeleqlkg qgksrlgdly eeemrelrrq vdqltndkar veverdnlae
181 dimrlreklq eemlqreeae ntlgsfrqdv dnaslarldl erkveslqee iaflkklhee
241 eiqelqaqiq eqhvqidvdv skpdltaalr dvrqqyesva aknlqeaeew ykskfadlse
301 aanrnndalr qakqesteyr rqvqsltcev dalkgtnesl erqmremeen faveaanyqd
361 tigrlqdeiq nmkeemarhl reyqdllnvk maldieiaty rkllegeesr islplpnfss
421 lnlretnlds lplvdthskr tlliktvetr dgqvinetsq hhddle
```

SEQ ID No. 1 is a 466 amino acid human vimentin protein. This sequence is identified as Accession No. P08670 in the SwissProt database, version updated Jan. 23, 2007. Human vimentin sequences are also detailed in Ferrari, S. et al., Mol. Cell Biol. 6:3614-3620, 1986; Deloukas, P. et al., Nature, 429:375-381, 2004; Gerhard et al., Genome Res., 14:2121-2127, 2004; and Sommers, C. L. et al., Cancer Res. 49:4258-4263, 1989.

```
SEQ ID No. 2 Annexin A5
    1 maqvlrgtvt dfpgfderad aetlrkamkg lgtdeesilt lltsrsnaqr qeisaafktl
  61 fgrdllddlk seltgkfekl ivalmkpsrl ydayelkhal kgagtnekvl teiiasrtpe
121 elraikqvye eeygssledd vvgdtsgyyq rmlvvllqan rdpdagidea qveqdaqalf
181 gagelkwgtd eekfitifgt rsvshlrkvf dkymtisgfq ieetidrets gnleqlllav
241 vksirsipay laetlyyamk gagtddhtli rvmvsrseid lfnirkefrk nfatslysmi
301 kgdtsgdykk allllcgedd
```

SEQ ID No. 2 is a 320 amino acid human annexin 5 protein. This sequence is identified as Accession No. P08758 in the SwissProt database, version updated Jan. 23, 2007. Human annexin 5 sequences are also detailed in Funakoshi, T. et al., Biochemistry, 26:8087-8092, 1987 and Fernandez, et al., Gene, 149:253-260, 1994.

```
SEQ ID No. 3 Annexin A5
      1 ggggagtcta ggtgcagctg ccggatcctt cagcgtctgc atctcggcgt cgccccgcgt
    61 accgtcgccc ggctctccgc cgctctcccg gggtttcggg gcacttgggt cccacagtct
  121 ggtcctgctt caccttcccc tgacctgagt agtcgccatg gcacaggttc tcagaggcac
  181 tgtgactgac ttccctggat ttgatgagcg ggctgatgca gaaactcttc ggaaggctat
  241 gaaaggcttg ggcacagatg aggagagcat cctgactctg ttgacatccc gaagtaatgc
  301 tcagcgccag gaaatttctg cagcttttaa gactctgttt ggcagggatc ttctggatga
  361 cctgaaatca gaactaactg gaaaatttga aaaattaatt gtggctctga tgaaaccctc
  421 tcggctttat gatgcttatg aactgaaaca tgccttgaag ggagctggaa caaatgaaaa
  481 agtactgaca gaaattattg cttcaaggac acctgaagaa ctgagagcca tcaaacaagt
  541 ttatgaagaa gaatatggct caagcctgga agatgacgtg gtgggggaca cttcagggta
  601 ctaccagcgg atgttggtgg ttctccttca ggctaacaga gaccctgatg ctggaattga
  661 tgaagctcaa gttgaacaag atgctcaggc tttatttcag gctggagaac ttaaatgggg
  721 gacagatgaa gaaaagttta tcaccatctt tggaacacga agtgtgtctc atttgagaaa
  781 ggtgtttgac aagtacatga ctatatcagg atttcaaatt gaggaaacca ttgaccgcga
  841 gacttctggc aatttagagc aactactcct tgctgttgtg aaatctattc gaagtatacc
  901 tgcctacctt gcagagaccc tctattatgc tatgaaggga gctgggacag atgatcatac
  961 cctcatcaga gtcatggttt ccaggagtga gactgatctg tttaacatca ggaaggagtt
1021 taggaagaat tttgccacct ctctttattc catgattaag ggagatacat ctggggacta
1081 taagaaagct cttctgctgc tctgtggaga agatgactaa cgtgtcacgg ggaagagctc
1141 cctgctgtgt gcctgcacca ccccactgcc ttccttcagc acctttagct gcatttgtat
1201 gccagtgctt aacacattgc cttattcata ctagcatgct catgaccaac acatacacgt
1261 catagaagaa aatagtggtg cttctttctg atctctagtg gagatctctt tgactgctgt
1321 agtactaaag tgtacttaat gttactaagt ttaatgcctg gccattttcc atttatatat
1381 attttttaag aggctagagt gcttttagcc ttttttaaaa actccattta tattacattt
1441 gtaaccatga tactttaatc agaagcttag ccttgaaatt gtgaactctt ggaaatgtta
1501 ttagtgaagt tcgcaactaa actaaacctg taaaattatg atgattgtat tcaaaagatt
1561 aatgaaaaat aaacatttct gtccccctga taaaaaaaaa aaaaaaaaaa aaaaaaaaa
```

SEQ ID No. 3 is a 1619 bp mRNA wherein the coding sequence of human annexin 5 extends from bp 158-1120. This sequence is identified as Accession No. BC018671 in the GenBank database, version BC018671.1. Human annexin 5 encoding nucleic acid sequences are also detailed in Strausberg et al., PNAS, 99:16899-16903, 2002; and Fernandez, et al., Gene, 149:253-260, 1994.

```
SEQ ID No. 4 6-phosphogluconolactonase
    1 maapapglis vtsssqelga alaqlvagra acclagarar falglsggsl vsmlarelpa
  61 avapagpasl arwtlgfcde rlvpfdhaes tyglyrthll srlpipesqv itinpelpve
121 eaaedyakkl rgafqgdsip vfdllilgvg pdghtcslfp dhpllqerek ivapisdspk
181 pppqrvtltl pvlnaartvi fvatgegkaa vlkriladqe enplpaalvq phtgklcwfl
241 deaaarlltv pfekhstl
```

SEQ ID No. 4 is a 258 amino acid human 6-phosphogluconolactonase protein. This sequence is identified as Accession No. 095336 in the SwissProt database, version updated May 30, 2000. Human 6-phosphogluconolactonase sequences are also detailed in Gerhard, D. S. et al., Genome Res., 14:2121-2127, 2004.

```
SEQ ID No. 5 6-phosphogluconolactonase
      1 ctcctccccg ccgccgccct cgtcatggcc gcgccggccc cgggcctcat ctcggtgttc
    61 tcgagttccc aggagctggg tgcggcgcta gcgcagctgg tggcccagcg cgcagcatgc
  121 tgcctggcag gggcccgcgc ccgtttcgcg ctcggcttgt cgggcgggag cctcgtctcg
  181 atgctagccc gcgagctacc cgccgccgtc gcccctgccg ggccagctag cttagcgcgc
  241 tggacgctgg gcttctgcga cgagcgcctc gtgcccttcg atcacgccga gagcacgtac
  301 ggcctctacc ggacgcatct tctctccaga ctgccgatcc cagaaagcca ggtgatcacc
  361 attaaccccg agctgcctgt ggaggaggcg gctgaggact acgccaagaa gctgagacag
  421 gcattccaag gggactccat cccggttttc gacctgctga tcctgggggt gggccccgat
  481 ggtcacacct gctcactctt cccagaccac cccctcctac aggagcggga gaagattgtg
  541 gctcccatca gtgactcccc gaagccaccg ccacagcgtg tgaccctcac gctacctgtc
  601 ctgaatgcag cacgaactgt catctttgtg gcaactggag aaggcaaggc agctgttctg
  661 aagcgcattt tggaggacca ggaggaaaac ccgctgcccg ccgccctggt ccagccccac
  721 accgggaaac tgtgctggtt cttggacgag gcggccgccc gcctcctgac cgtgcccttc
  781 gagaagcatt ccactttgta gctggccaga gggacgccgc agctgggacc aggcacgcgg
  841 cccatggggc tgggcccctg ctggccgcca ctctccgggc tctcctttca aaaagccacg
  901 tcgtgctgct gctggaagcc aacagcctcc ggccagcagc cctacccggg gctcaacaca
  961 caggctgtgg ctctggacat ccggatatta aaaggagcgt tgctggaaaa aaaaaaaaaa
1021 aaaaaaaaaa aaaa
```

SEQ ID No. 5 is a 1034 nucleotide human 6-phosphogluconolactonase encoding sequence. This sequence is identified as Accession No. BC014006 in the GenBank database, version BC014006.2. In this sequence, the coding region extends from bp 25-801. Human 6-phosphogluconolactonase encoding sequences are also detailed in Strausberg et al., PNAS, 99:16899-16903, 2002.

Any patents or publications mentioned in this specification are incorporated herein by reference to the same extent as if each individual publication is specifically and individually indicated to be incorporated by reference. U.S. Provisional Patent Application 60/773,926, filed Feb. 16, 2006, is incorporated by reference herein in its entirety.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

We claim:

1. A method of detecting a form of chronic lymphocytic leukemia in a subject, comprising:
assaying a sample obtained from the subject having or suspected of having chronic lymphocytic leukemia for a marker selected from the group consisting of: a vimentin cleavage product, annexin 5,6-phosphogluconolactonase, and a combination of any of these to obtain a first assay result, wherein the assay result is indicative of a form of chronic lymphocytic leukemia in the subject.

2. The method of claim 1, wherein detecting the cleavage product comprises contacting the sample with a vimentin cleavage product binding agent.

3. The method of claim 1, further comprising detecting uncleaved vimentin.

4. The method of claim 1, wherein assaying the sample comprises detecting a substance selected from the group consisting of: an annexin 5 protein, an annexin 5 nucleic acid, and a combination thereof.

5. The method of claim 1, wherein assaying the sample comprises detecting a substance selected from the group consisting of: a 6-phosphogluconolactonase protein, a 6-phosphogluconolactonase nucleic acid, and a combination thereof.

6. The method of claim 1, wherein the assaying comprises a technique selected from the group consisting of: ELISA, flow cytometry, immunoblot, immunoprecipitation, immunocytochemistry, radioimmunoassay, RT-PCR, Northern blot hybridization, dot blot hybridization, RNAse protection, mass spectrometry, enzyme assay, and a combination of any of these.

7. The method of claim 1 wherein the first assay result is compared with a standard.

8. The method of claim 1, further comprising assaying a sample obtained from the subject having or suspected of having chronic lymphocytic leukemia for a marker selected from the group consisting of: beta-2-microglobulin, immunoglobulin variable region heavy chain gene mutation, ZAP-70, CD-38, lipoprotein lipase, a chromosomal abnormality associated with chronic lymphocytic leukemia, and a combination of any of these to obtain a second assay result.

9. The method of claim 1, wherein the sample comprises a normal or abnormal B-cell.

10. A method of detecting a form of chronic lymphocytic leukemia in a subject, comprising:
contacting a sample obtained from the subject having or suspected of having chronic lymphocytic leukemia with a vimentin cleavage product binding agent under binding conditions;
assaying the sample to detect the presence of a complex of a vimentin cleavage product binding agent and a vimentin cleavage product to determine the presence of the vimentin cleavage product; wherein detection of the presence of the vimentin cleavage product is indicative of a form of chronic lymphocytic leukemia in the subject, thereby detecting a form of chronic lymphocytic leukemia in the subject.

11. The method of claim 10, further comprising contacting the sample obtained from the subject having or suspected of having chronic lymphocytic leukemia with a total vimentin binding agent.

12. The method of claim 11 wherein detection of a ratio of the vimentin cleavage product to total vimentin of about 0.33 or greater is indicative of favorable prognosis of chronic lymphocytic leukemia in the subject.

13. The method of claim 10 wherein the vimentin cleavage product binding agent and the total vimentin binding agent are identical.

14. The method of claim 10 wherein the vimentin cleavage product binding agent is an antibody recognizing an epitope common to the vimentin cleavage product and uncleaved vimentin.

15. The method of claim 10 wherein the vimentin cleavage product binding agent recognizes an epitope specific to the vimentin cleavage product and does not substantially bind uncleaved vimentin.

16. The method of claim 10 wherein the vimentin cleavage product has a molecular weight in the range of about 52-54 kDa.

17. A method of detecting a form of chronic lymphocytic leukemia in a subject, comprising:
assaying a sample obtained from the subject having or suspected of having chronic lymphocytic leukemia for a marker selected from the group consisting of: annexin 5,6-phosphogluconolactonase, and a combination of these to obtain a first assay result; and
comparing the first assay result with a standard, wherein detection of enrichment of annexin 5 and/or 6-phosphogluconolactonase is indicative of a form of chronic lymphocytic leukemia having a poor prognosis, thereby detecting a form of chronic lymphocytic leukemia in the subject.

18. A kit for detection of a form of chronic lymphocytic leukemia in a subject, comprising:
a binding agent for substantially specific binding to a substance selected from the group consisting of: a vimentin cleavage product, uncleaved vimentin, annexin 5,6-phosphogluconolactonase, an annexin 5 nucleic acid, a 6-phosphogluconolactonase nucleic acid, and a combination of any of these; and
instructions for use in detection of a form of chronic lymphocytic leukemia in a subject.

19. The kit of claim 18 further comprising a binding agent for specifically detecting beta-2-microglobulin, immunoglobulin variable region heavy chain gene mutation, ZAP-70, CD-38, lipoprotein lipase, a chromosomal abnormality associated with chronic lymphocytic leukemia, and a combination of any of these.
